(19) United States
(12) Patent Application Publication
(10) Pub. No.: US 2003/0119004 A1
(43) Pub. Date: Jun. 26, 2003

(54) Methods for quantitating nucleic acids using coupled ligation and amplification

(76) Inventors: H. Michael Wenz, Redwood City, CA (US); Gary P. Schroth, San Ramon, CA (US); Caifu Chen, Palo Alto, CA (US)

Correspondence Address: Finnegan, Henderson, Farabow,, Garret & Dunner, L.L.P., 1300 I Street, N.W., Washington, DC, 20005-3315, US

(21) Appl. No.: 10/011,993

(22) Filed: Dec. 5, 2001

(51) Int. Cl. C12Q 1/68; C07H 21/04; C12P 19/34

(52) U.S. Cl. 435/6; 435/91.2; 536/24.3

(57) ABSTRACT

The present invention relates to methods and kits for quantitating target nucleic acid sequences using coupled ligation and amplification. The invention also relates to methods, reagents, and kits that employ addressable-support specific portions.

### FIELD OF THE INVENTION

The present invention generally relates to quantifying nucleic acid levels using coupled ligation and amplification reactions. The invention also relates to methods and kits for quantifying levels of nucleic acid.

### BACKGROUND OF THE INVENTION

An organism's genetic makeup is determined by the genes contained within the genome of that organism. Genes are composed of long strands or deoxyribonucleic acid (DNA) polymers that encode the information needed to make proteins. Properties, capabilities, and traits of an organism often are related to the types and amounts of proteins that are, or are not, being produced by that organism.

A protein can be produced from a gene as follows. First, the DNA of the gene that encodes a protein, for example, protein “X”, is converted into ribonucleic acid (RNA) by a process known as “transcription.” During transcription, a single-stranded complementary RNA copy of the gene is made. Next, this RNA copy, referred to as protein X messenger RNA (mRNA), is used by the cell's biochemical machinery to make protein X, a process referred to as “translation.” Basically, the cell's protein manufacturing machinery binds to the mRNA, “reads” the RNA code, and “translates” it into the amino acid sequence of protein X. In summary, DNA is transcribed to make mRNA, which is translated to make proteins.

The amount of protein X that is produced by a cell often is largely dependent on the amount of protein X mRNA that is present within the cell. The amount of protein X mRNA within a cell is due, at least in part, to the degree to which gene X is expressed. Whether a particular gene is expressed, and if so, to what level, may have a significant impact on the organism.

### SUMMARY OF THE INVENTION

According to certain embodiments, methods for quantitating at least one target nucleic acid sequence in a sample are provided. In certain embodiments, the methods comprise combining at least one target nucleic acid sequence with a probe set for each target nucleic acid sequence to form a ligation reaction mixture. In certain embodiments, the probe set comprises (a) at least one first probe, comprising a first target-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one probe in each probe set further comprises at least one addressable support-specific portion. In certain embodiments, when the at least one first probe comprises the at least one addressable support-specific portion, the at least one first probe further comprises a 5′ primer-specific portion. In certain embodiments, the at least one addressable support-specific portion is located between the primer-specific portion and the target-specific portion of the at least one probe in each probe set.

In certain embodiments, the methods further comprise subjecting the ligation reaction mixture to at least one cycle of ligation, wherein adjacently hybridized probes are ligated to form a ligation product comprising the first and second target specific portions, the at least one addressable support-specific portion, and the 3′ primer-specific portion. In certain embodiments, the methods further comprise combining the ligation product with at least one primer set comprising at least one second primer comprising a sequence complementary to the 3′ primer-specific portion of the ligation product and a DNA polymerase to form a first amplification reaction mixture. In certain embodiments, the methods further comprise subjecting the first amplification reaction mixture to at least one cycle of amplification to generate a first amplification product. In certain embodiments, the methods further comprise detecting the first amplification product or a portion of the first amplification product using the at least one addressable support-specific portion. In certain embodiments, the methods further comprise quantitating the at least one target nucleic acid sequence.

According to certain embodiments, methods for quantitating at least one target nucleic acid sequence in a sample are provided. In certain embodiments, the methods comprise combining at least one target nucleic acid sequence with a probe set for each target nucleic acid sequence to form a ligation reaction mixture. In certain embodiments, the probe set comprises (a) at least one first probe, comprising a first target-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one probe in each probe set further comprises a promoter or its complement. In certain embodiments, at least one probe in each probe set further comprises at least one addressable support-specific portion. In certain embodiments, when the at least one first probe comprises the at least one addressable support-specific portion, the at least one first probe further comprises a 5′ primer-specific portion. In certain embodiments, the at least one addressable support-specific portion is located between the primer-specific portion and the target-specific portion of the at least one probe in each probe set.

In certain embodiments, the methods further comprise subjecting the ligation reaction mixture to at least one cycle of ligation, wherein adjacently hybridized probes are ligated to form a ligation product comprising the first and second target specific portions, the at least one addressable support-specific portion, the 3′ primer-specific portion, and the promoter or its compliment. In certain embodiments, the methods further comprise combining the ligation product with at least one primer set comprising at least one second primer comprising a sequence complementary to the 3′ primer-specific portion of the ligation product and a DNA polymerase to form a first amplification reaction mixture. In certain embodiments, the methods further comprise subjecting the first amplification reaction mixture to at least one cycle of amplification to generate a first amplification product comprising the promoter. In certain embodiments, the methods further comprise combining the first amplification product with an RNA polymerase and a ribonucleoside triphosphate solution comprising at least one of rATP, rCTP, rGTP, or rUTP, to form a transcription reaction mixture. In certain embodiments, the methods further comprise incubating the transcription reaction mixture under appropriate conditions to generate an RNA transcription product. In certain embodiments, the methods further comprise detecting the RNA transcription product or a portion of the RNA transcription product using the at least one addressable support-specific portion. In certain embodiments, the methods further comprise quantitating the at least one target nucleic acid sequence.

According to certain embodiments, methods for quantitating at least one target nucleic acid sequence in a sample are provided. In certain embodiments, the methods comprise combining at least one target nucleic acid sequence with a probe set for each target nucleic acid sequence to form a ligation reaction mixture. In certain embodiments, the probe set comprises (a) a first probe, comprising a first target-specific portion and a 5′ primer-specific portion, and (b) a second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one probe in each probe set further comprises at least one addressable support-specific portion located between the primer-specific portion and the target-specific portion of the at least one probe in each probe set.

In certain embodiments, the methods further comprise subjecting the ligation reaction mixture to at least one cycle of ligation, wherein adjacently hybridized probes are ligated to form a ligation product comprising the 5′ primer specific portion, the first and second target specific portions, the at least one addressable support-specific portion, and the 3′ primer-specific portion. In certain embodiments, the methods further comprise combining the ligation product with at least one primer set comprising (a) at least one primer set comprising: (i) at least one first primer comprising the sequence of the 5′ primer-specific portion of the ligation product, and (ii) at least one second primer comprising a sequence complementary to the 3′ primer-specific portion of the ligation product; and (b) a DNA polymerase; to form a first amplification reaction mixture. In certain embodiments, the methods further comprise subjecting the first amplification reaction mixture to at least one cycle of amplification to generate a first amplification product. In certain embodiments, the methods further comprise combining the first amplification product with either at least one first primer, or at least one second primer for each primer set, but not both first and second primers, to form a second amplification reaction mixture. In certain embodiments, the methods further comprise subjecting the second amplification reaction mixture to at least one cycle of amplification to generate a second amplification product. In certain embodiments, the methods further comprise detecting the first amplification product or a portion of the first amplification product using the at least one addressable support-specific portion. In certain embodiments, the methods further comprise quantitating the at least one target nucleic acid sequence.

According to certain embodiments, kits for quantitating at least one target nucleic acid sequence in a sample are provided. In certain embodiments, the kits comprise at least one probe set comprising (a) at least one first probe, comprising a first target-specific portion and a 5′ primer-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one probe in each probe set further comprises at least one addressable support-specific portion located between the primer-specific portion and the target-specific portion of the at least one probe in each probe set.

According to certain embodiments, kits for quantitating at least one target nucleic acid sequence in a sample are provided. In certain embodiments, the kits comprise at least one probe set comprising (a) at least one first probe, comprising a first target-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one second probe in each probe set further comprises at least one addressable support-specific portion located between the primer-specific portion and the target-specific portion of the at least one second probe in each probe set.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The skilled artisan will understand that the drawings, described below, are for illustration purposes only. The figures are not intended to limit the scope of the invention in any way.

FIG. 1. Schematic diagram depicting a general overview of certain exemplary embodiments of the invention.

FIG. 2. Schematic showing an exemplary embodiment of certain embodiments comprising ligation coupled to primer extension amplification.

FIG. 3 depicts exemplary embodiments of the invention comprising ligation coupled with PCR-based amplification, wherein the exemplary target nucleic acid sequence is an mRNA in the sample.

FIG. 4 depicts exemplary embodiments comprising a ligation reaction coupled to amplification using RNA polymerase to generate RNA transcription products.

FIG. 5 schematically illustrates exemplary embodiments comprising ligation coupled to primer extension followed by transcription.

FIG. 6 is a schematic showing a probe set according to certain embodiments of the invention.

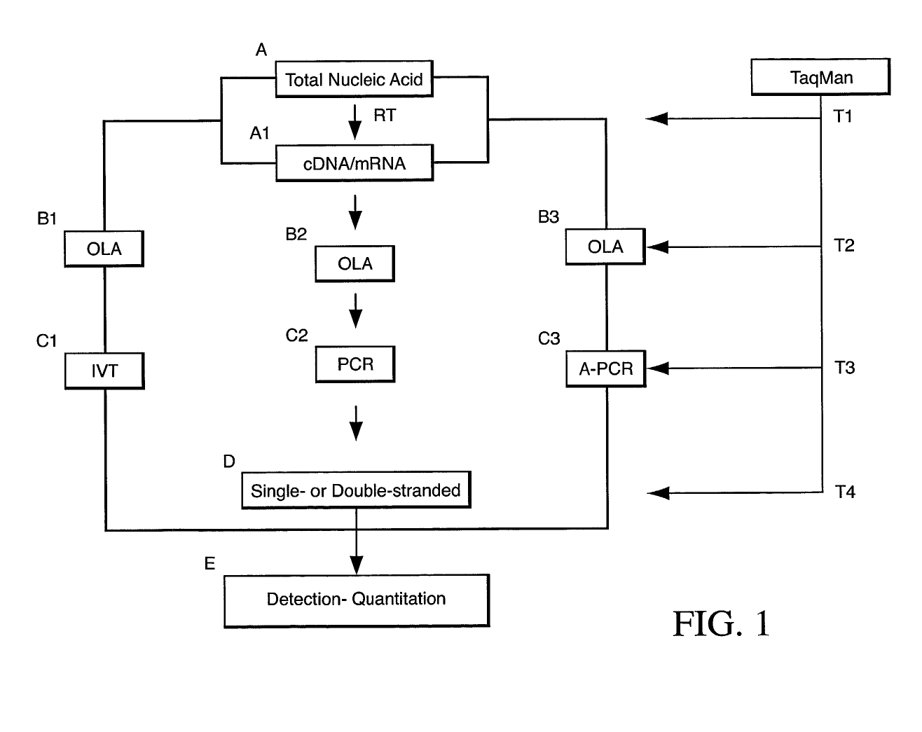

Each probe includes a portion that is complementary or substantially complementary to the target (the “target-specific portion,” T-SP) and a portion that is complementary to or has the same sequence as a primer (the “primer-specific portion,” P-SP). At least one probe in each probe set further comprises an addressable support-specific portion (AS-SP) that is located between the target-specific portion and the primer-specific portion (here, probe Z).

Each probe set comprises at least one first probe and at least one second probe that are designed to hybridize with the target with the 3′ end of the first probe (here, probe A) immediately adjacent to and opposing the 5′ end of the second probe (here, probe Z).

FIG. 7 depicts a method for differentiating between two potential alleles in a target locus using certain embodiments of the invention.

FIG. 7(a) shows: (i) a target-specific probe set comprising two first probes, A and B, that differ in their primer-specific portions and their pivotal complement (T on the A probe and C on the B probe), and one second probe, Z, comprising an addressable support-specific portion and a primer-specific portion, and (ii) a target sequence, comprising pivotal nucleotide A.

FIG. 7(b) shows the three probes annealed to the target. The target-specific portion of probe A is fully complementary with the 3′ target region including the pivotal nucleotide. The pivotal complement of probe B is not complementary with the 3′ target region. The target-specific portion of probe B, therefore, contains a base-pair mismatch at the 3′ end. The target-specific portion of probe Z is fully complementary to the 5′ target region.

FIG. 7(c) shows ligation of probes A and Z to form ligation product A-Z. Probes B and Z are not ligated together to form a ligation product due to the mismatched pivotal complement on probe B.

FIG. 7(d) shows denaturing the double-stranded molecules to release the A-Z ligation product and unligated probes B and Z.

FIG. 8 is a schematic depicting certain embodiments of the inventive methods.

FIG. 8(a) depicts a target sequence and a probe set comprising two first probes, A and B, that differ in their primer-specific portions and their pivotal complements (here, T at the 3′ end probe A and G at the 3′ end probe B), and one second probe, Z comprising the addressable support-specific portion (shown in wavy lines -vvvvv- upstream from primer-specific portion Z).

FIG. 8(b) depicts the A and Z probes hybridized to the target sequence under annealing conditions.

FIG. 8(c) depicts the ligation of the first and second probes in the presence of a ligation agent to form ligation product A-Z.

FIG. 8(d) depicts denaturing the ligation product:target complex to release a single-stranded ligation product; adding a primer set (PA*, PB*, and PZ), where the PA and PB primers comprise a reporter group (*); and annealing primer PZ to the ligation product.

FIG. 8(e) depicts the formation of a double-stranded nucleic acid product by extending the PZ primer in a template-dependent manner with a polymerase.

FIG. 8(f) depicts denaturing the double-stranded nucleic acid product to release two single-stranded molecules.

FIG. 8(g) shows the PA* and PZ primers annealed to their respective single-stranded molecules.

FIG. 8(h) shows both double-stranded amplification products.

FIG. 8(i) depicts both amplification products being denatured to release four single-stranded molecules including a single-stranded molecule comprising a reporter group, PA*.

FIG. 8(j) shows annealing the addressable support-specific portion of the single-stranded PA* amplification product to position 1 of the support.

FIG. 8(k) represents detecting the reporter group hybridized to position 1 of the support.

FIG. 9 depicts two or more ligation products comprising the same primer-specific portions and their respective primer sets.

FIG. 9(a) shows six ligation products and their respective primers. Each of the ligation products comprise a unique addressable support-specific portion (AS-SP). Two of the six ligation products comprise the same 5′ primer-specific portion and the same 3′ primer-specific portion, A and Z respectively. Consequently, only five primer sets (PA and PZ; PC and PX; PD and PW; PE and PV; and PF and PU) are required to amplify the six ligation products.

FIG. 9(b) shows six ligation products and their respective primers. Here most of the ligation products (4 of 6) comprise the same 5′ primer-specific portion and the same 3′ primer-specific portion, A and Z respectively. Consequently, only three primer sets (PA and PZ; PE and PV; and PF and PU) are required to amplify the six ligation products.

FIG. 9(c) shows six ligation products and their respective primers. Each of the six ligation products comprise unique addressable support-specific portions. All six ligation products comprise the same 5′ primer-specific portion and the same 3′ primer-specific portion, A and Z respectively. Consequently, only one primer set (PA and PZ) is required to amplify all six ligation products.

FIG. 10 depicts exemplary alternative splicing.

FIG. 11 depicts certain embodiments for identifying splice variants.

For identifying the splice variant including exon 1, exon 2, and exon 4, one employs a probe set that comprises two probes. One probe comprises PSPa, ASSP, and TSP, and the other probe comprises PSPb and SSP (corresponding to at least a portion of exon 2).

For identifying the splice variant including exon 1, exon 3, and exon 4, one employs a probe set that comprises two probes. One probe comprises PSPa, ASSP, and TSP, and the other probe comprises PSPc and SSP (corresponding to at least a portion of exon 3).

FIG. 12 graphically illustrates the amount of the four species of target nucleic sequences as discussed in Example 5 as quantitated by a TaqMan™ assay. FIG. 12A shows such results following at least one ligation reaction, comprising 100 femtomoles (fM) of each target nucleic acid sequence initially. FIG. 12B shows such results following at least one amplification reaction, which followed at least one ligation reaction, comprising 100 femtomoles (fM) of each target nucleic acid sequence initially.

FIG. 13 graphically illustrates the results of a target nucleic acid template quantitation, similar to that shown in FIG. 12, but wherein the four target nucleic acid species were initially present at concentrations of 1,000 fM (COX6b), 100 fM (RPS4x), 10 fM (GAPDH), or 0.1 fM (Beta-actin). FIG. 13A shows such results following at least one ligation reaction. FIG. 13B shows such results following at least one amplification reaction, which followed at least one ligation reaction.

FIG. 14 illustrates work of Example 6, which resulted in detection and quantitation of amplification products comprising at least one cyanine 3 (Cy3), using a microarray hybridization technique when 100 femtomoles (fM) of each target nucleic acid sequence was used. “PMT:600” refers to the voltage setting on the laser scanner used for imaging the microarrays.

FIG. 15 illustrates work of Example 6, which resulted in detection and quantitation of amplification products comprising at least one cyanine 3 (Cy3), using a microarray hybridization technique, when four target nucleic acid sequences were initially present at concentrations of 1,000 fM (COX6b), 100 fM (RPS4x), 10 fM (GAPDH), or 0.1 fM (Beta-actin).

### DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. U.S. patent application Ser. No. 09/584,905, filed May 30, 2000, and Ser. No. 09/724,755, filed Nov. 28, 2000, and Patent Cooperation Treaty Application No. PCT/US01/17329, filed May 30, 2001, are hereby expressly incorporated by reference in their entirety for any purpose.

Definitions

An “enzymatically active mutant or variant thereof,” when used in reference to an enzyme such as a polymerase or a ligase, means a protein with appropriate enzymatic activity. Thus, for example, but without limitation, an enzymatically active mutant or variant of a DNA polymerase is a protein that is able to catalyze the stepwise addition of appropriate deoxynucleoside triphosphates into a nascent DNA strand in a template-dependent manner. An enzymatically active mutant or variant differs from the “generally-accepted” or consensus sequence for that enzyme by at least one amino acid, including, but not limited to naturally-occurring or designed amino acid substitutions, deletions and insertions, provided that at least some catalytic activity is retained. Fragments, for example, but without limitation, proteolytic cleavage products are also encompassed by this term, provided that at least some enzyme catalytic activity is retained.

The skilled artisan will readily be able to measure catalytic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups. Protocols for such assays may be found, among other places, in Sambrook et al., Molecular Cloning, A Laboratory Manual, Cold Spring Harbor Press (1989), hereinafter “Sambrook et al.,” Sambrook and Russell, Molecular Cloning, Third Edition, Cold Spring Harbor Press (2000), (hereafter “Sambrook and Russell”), Ausbel et al., Current Protocols in Molecular Biology (1993) including supplements through April 2001, John Wiley & Sons, (hereafter “Ausbel et al.”)

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine. The term “nucleotide” as used herein refers to a phosphate ester of a nucleoside, e.g., a triphosphate ester, wherein the most common site of esterification is the hydroxyl group attached to the C-5 position of the pentose. See, e.g., Kornberg and Baker, DNA Replication, 2nd Ed. (Freeman, San Francisco, 1992). The term “nucleoside” as used herein refers to a set of compounds including both nucleosides and nucleotides.

The term “polynucleotide” means polymers of nucleotide monomers, including analogs of such polymers, including double- and single-stranded deoxyribonucleotides, ribonucleotides, α-anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H⁺, NH₄⁺, Na⁺, if such counterions are present. Whenever a polynucleotide is represented by a sequence of letters, such as “ATGCCTG,” it will be understood that: (i) the nucleotides are in 5′ to 3′ order from left to right unless otherwise noted or it is apparent to the skilled artisan from the context that the converse was intended; and (ii) that “A” denotes deoxyadenosine, “C” denotes deoxycytidine, “G” denotes deoxyguanosine, and “T” denotes deoxythymidine;. Descriptions of how to synthesize oligonucleotides can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; and 5,262,530. Oligonucleotides can be of any length. In certain embodiments, the oligonucleotides may be 12 to 40 nucleotides in length. In certain embodiments, the oligonucleotides may be 15 to 35 nucleotides in length. In certain embodiments, the oligonucleotides may be 17 to 25 nucleotides in length.

“Analogs” in reference to nucleosides and/or polynucleotides comprise synthetic analogs having modified nucleobase portions, modified pentose portions and/or modified phosphate portions, and, in the case of polynucleotides, modified internucleotide linkages, as described generally elsewhere (e.g., Scheit, Nucleotide Analogs (John Wiley, New York, (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH₄⁺, Na⁺, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. According to certain embodiments, nucleobase analogs are iso-C and iso-G nucleobase analogs available from Sulfonics, Inc., Alachua, Fla. (e.g., Benner, et al., U.S. Pat. No. 5,432,272) or LNA analogs (e.g., Koshkin et al., Tetrahedron 54:3607-30 (1998)). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include, but are not limited to, phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). Internucleotide linkage analogs include, but are not limited to, peptide nucleic acid (PNA), morpholidate, acetal, and polyamide-linked heterocycles. In certain embodiments, one may use a class of polynucleotide analogs where a conventional sugar and internucleotide linkage has been replaced with a 2-aminoethylglycine amide backbone polymer is PNA (e.g., Nielsen et al., Science, 254:1497-1500 (1991); Egholm et al., J. Am. Chem. Soc., 114:1895-1897 (1992)).

The term “reporter group” as used herein refers to any tag, label, or identifiable moiety. The skilled artisan will appreciate that many reporter groups may be used in the present invention. For example, reporter groups include, but are not limited to, fluorophores, radioisotopes, chromogens, enzymes, antigens, heavy metals, dyes, magnetic probes, phosphorescence groups, chemiluminescent groups, and electrochemical detection moieties. Exemplary fluorophores that are used as reporter groups include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, and 6-Fam™ are all available from Applied Biosystems, Foster City, Calif.) Exemplary radioisotopes include, but are not limited to, ³²P, ³³P, and ³⁵S. Reporter groups also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label. The skilled artisan will appreciate that, in certain embodiments, one or more of the primers, probes, deoxyribonucleotide triphosphates, ribonucleotide triphosphates disclosed herein may further comprise one or more reporter groups. Detailed protocols for methods of attaching reporter groups to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

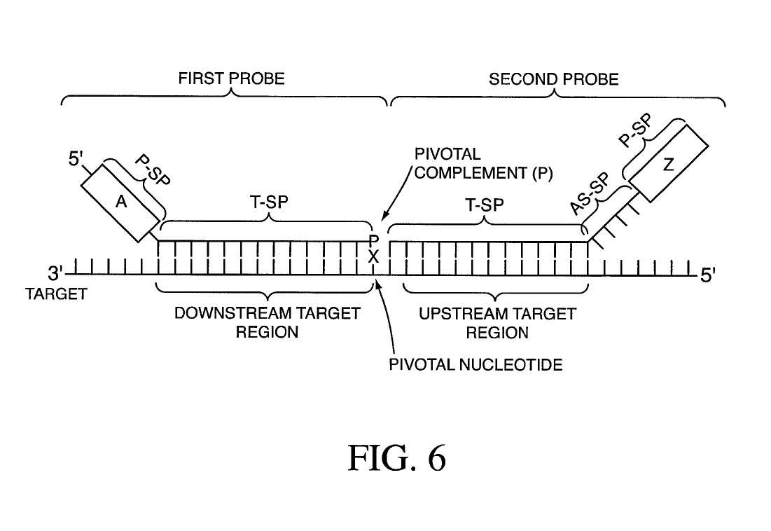

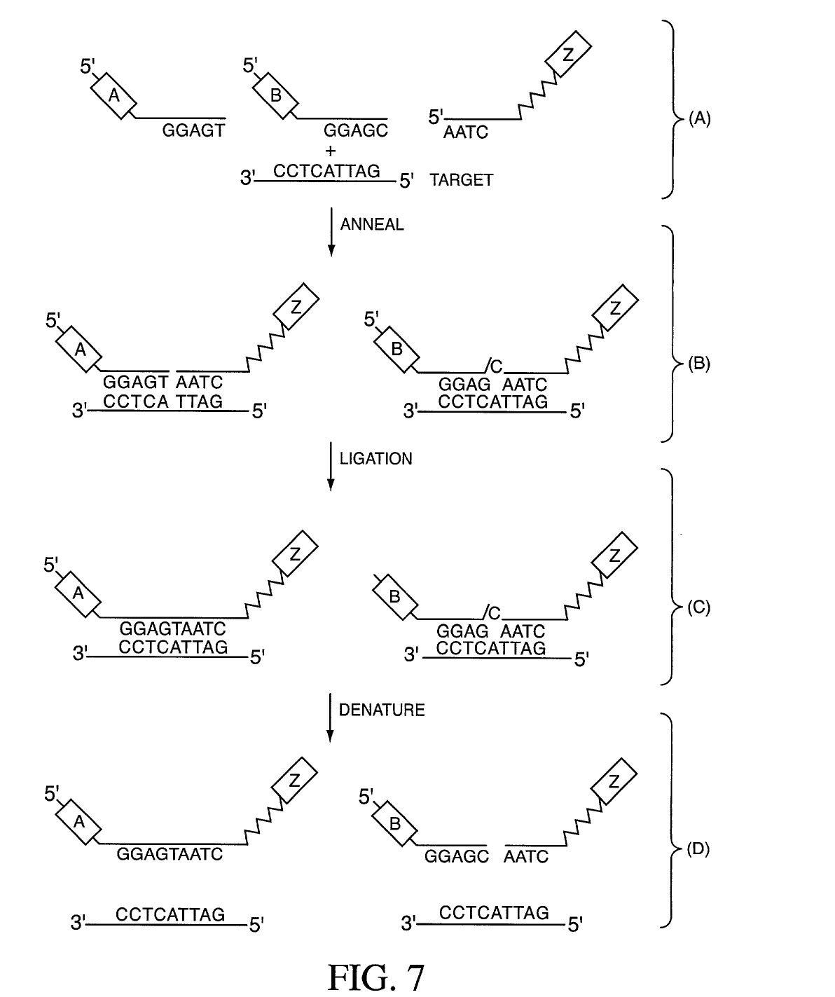

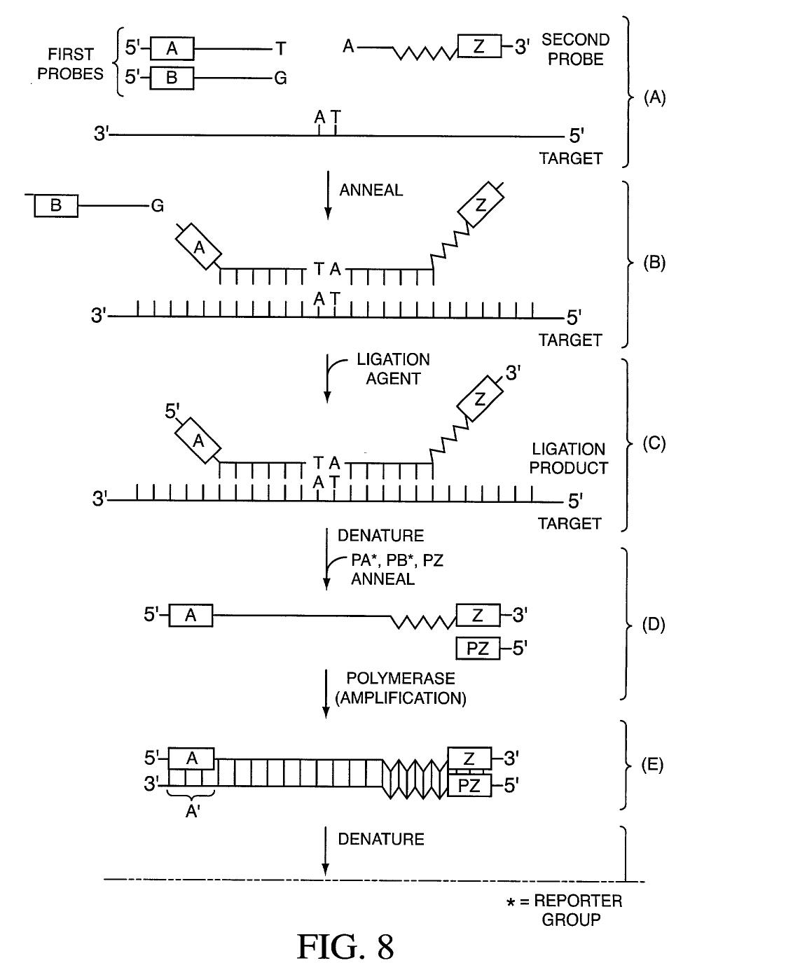

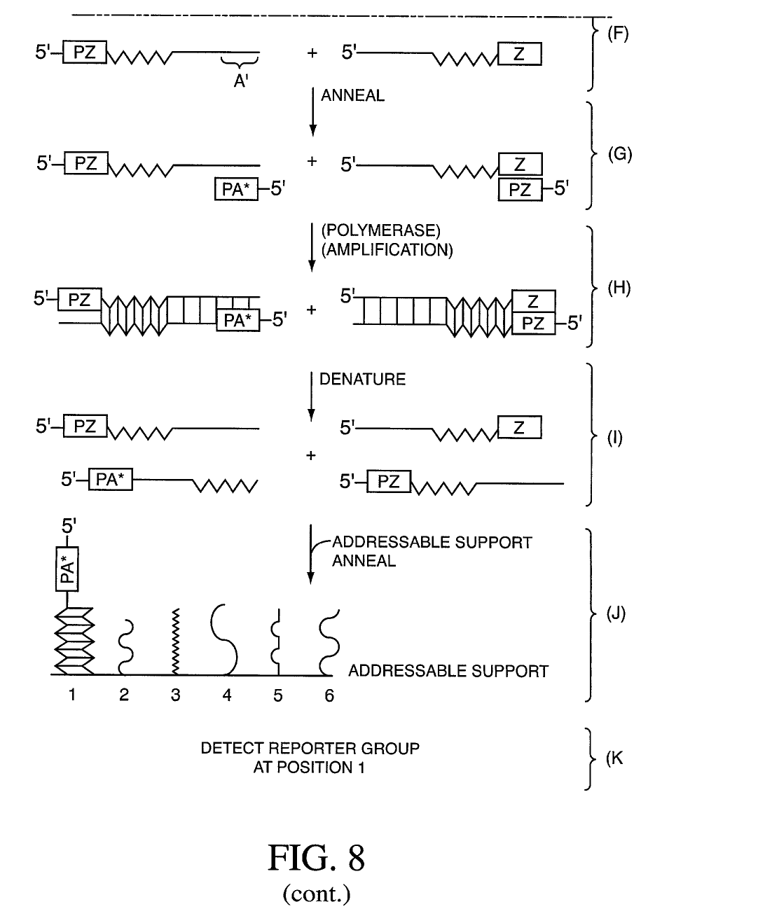

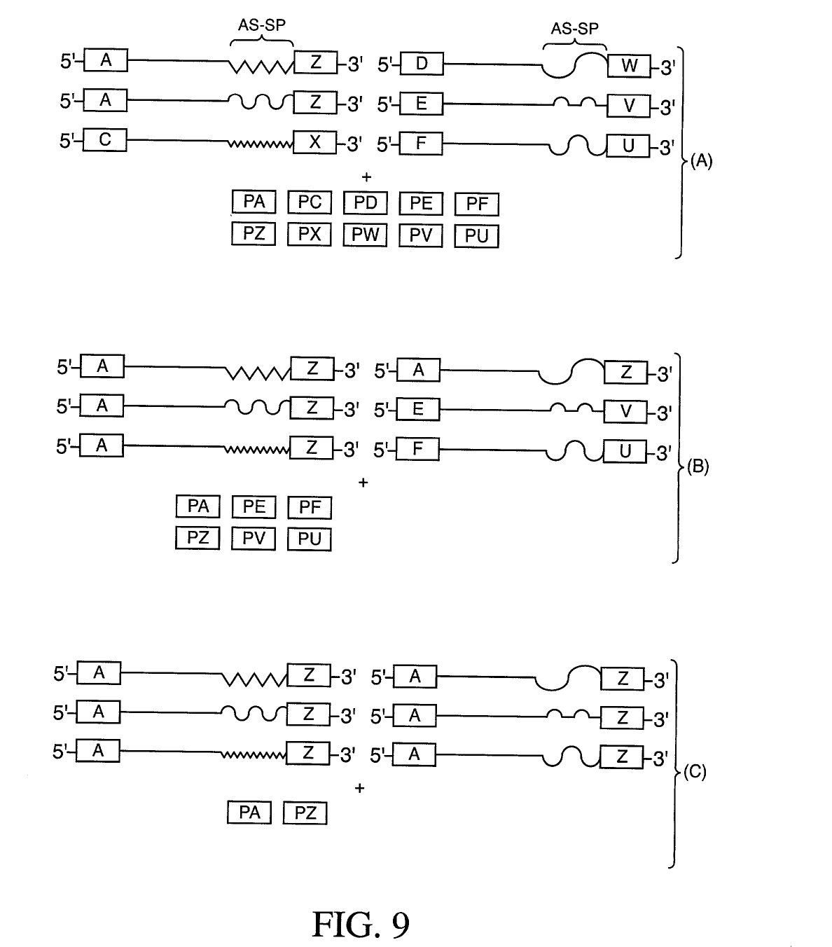

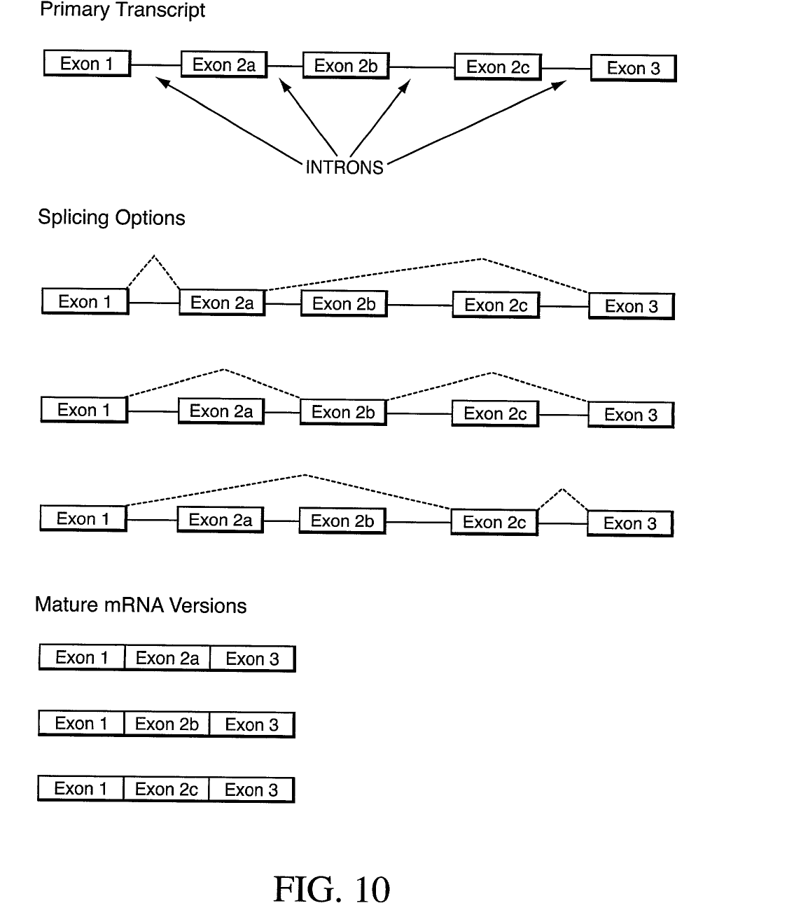

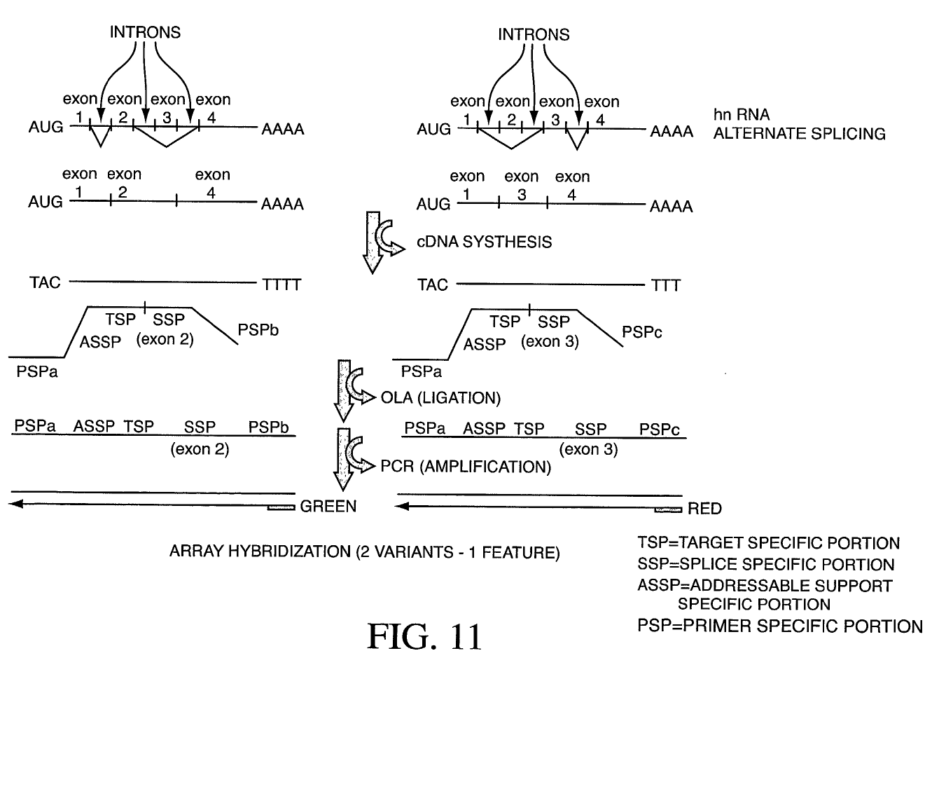

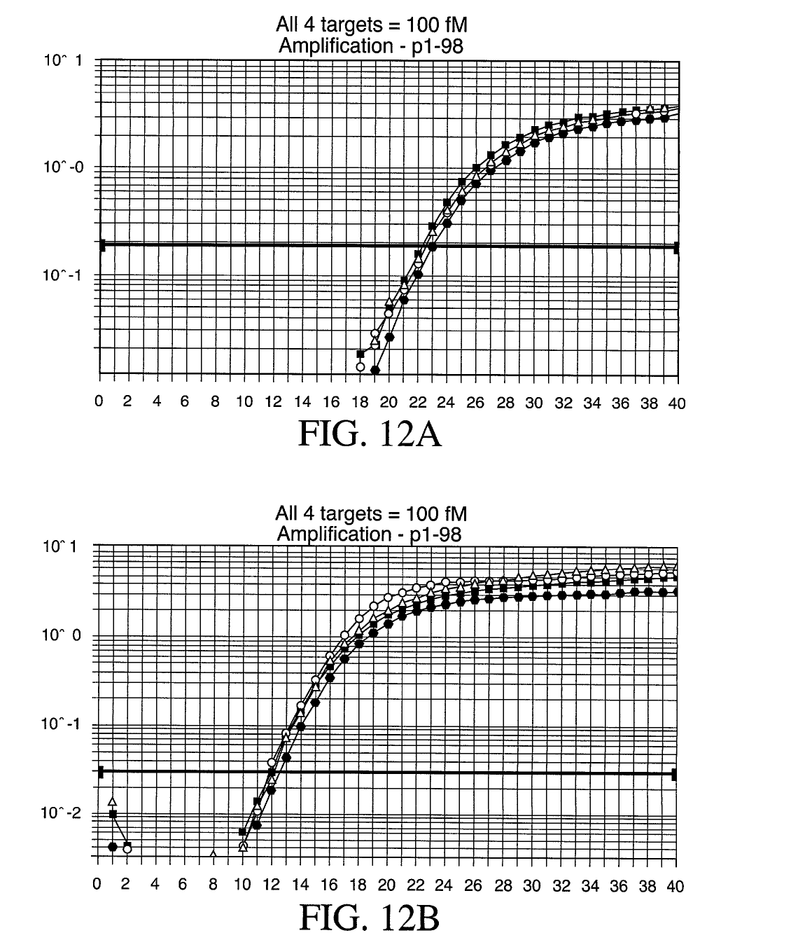

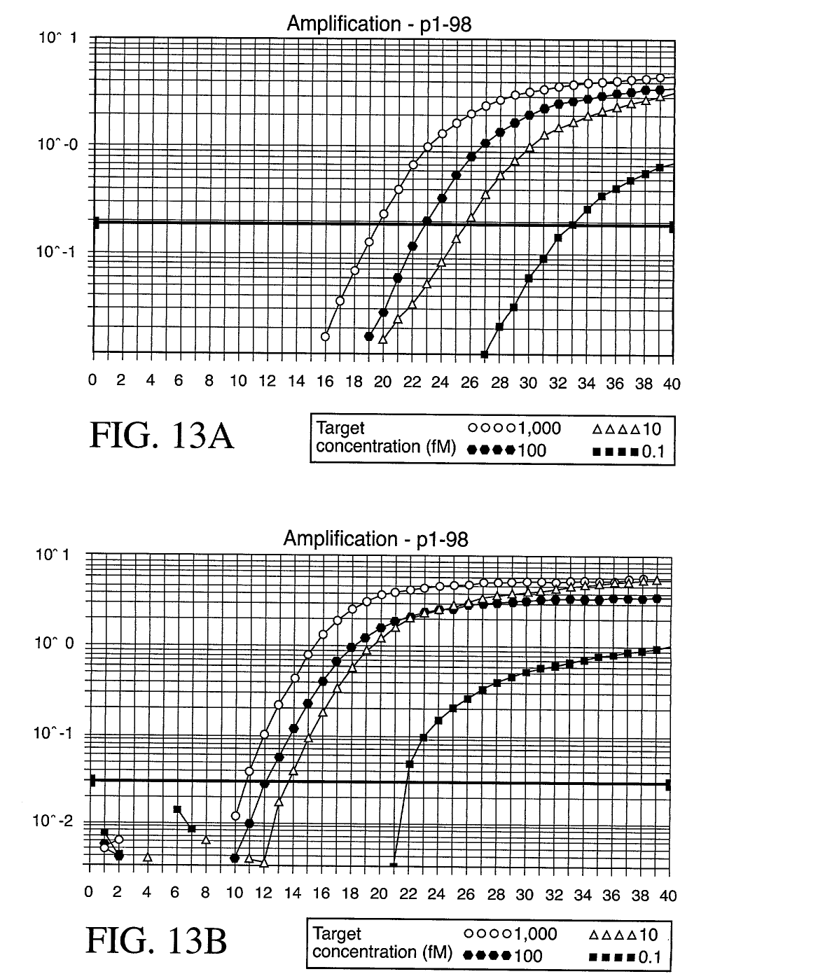

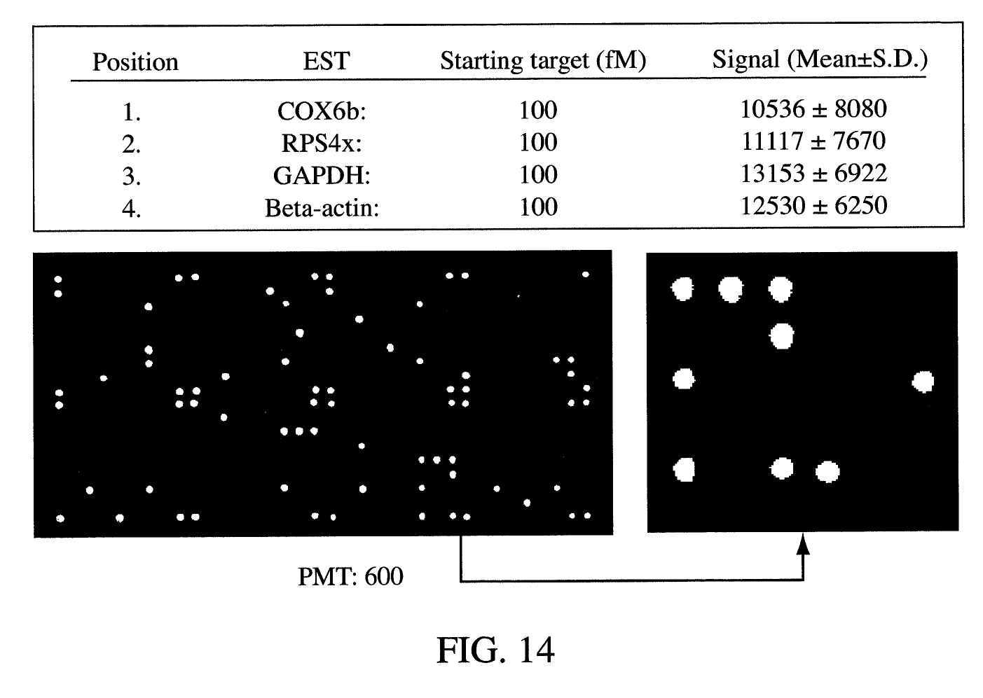

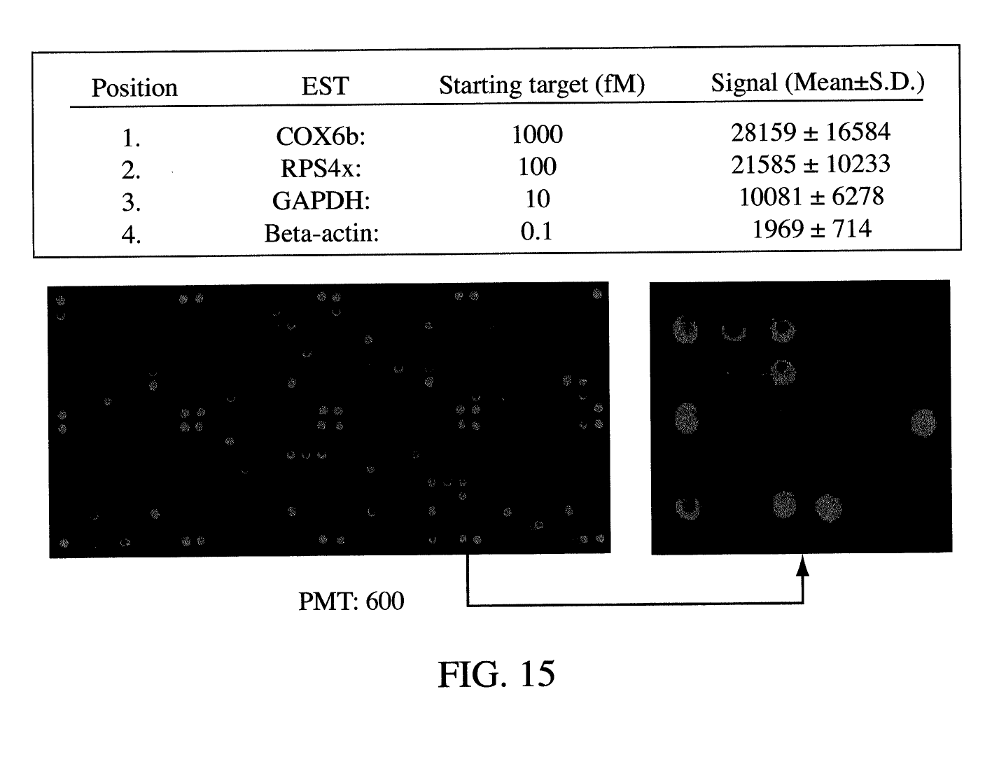

A “target” or “target nucleic acid sequence” according to the present invention comprises a specific nucleic acid sequence that is to be detected and quantified. The term target nucleic acid sequence encompasses both DNA and RNA. The person of ordinary skill will appreciate that while the target nucleic acid sequence may be described as a single-stranded molecule, the complement of that single-stranded molecule, or a double-stranded target nucleic acid molecule may also serve as a target nucleic acid sequence. For example, but without limitation, DNA molecules are typically double-stranded and either or both strands may be used as a nucleic acid target sequence. In certain embodiments, a target sequence comprises an upstream or 5′ region, a downstream or 3′ region, and a “pivotal nucleotide” located between the upstream region and the downstream region (see, e.g., FIG. 6). The pivotal nucleotide is the nucleotide being detected by the probe set and may represent, for example, without limitation, a single polymorphic nucleotide in a multiallelic target locus.

The term “target nucleic acid sequence” generally refers to a nucleotide sequence that, under appropriate conditions, directs the synthesis of a new nucleic sequence, typically with a DNA polymerase, a transcriptase, or an RNA polymerase. The target nucleic acid sequence may be the actual target nucleic acid present in a specimen or starting material, or the like, or it may be a counterpart of that sequence, such as a cDNA derived from a target RNA sequence present in the starting material. In certain embodiments, the target nucleic acid sequence may comprise single- or double-stranded DNA; cDNA, either single-stranded or double-stranded, and including both DNA:DNA and DNA:RNA hybrids; and RNA, including, but not limited to, mRNA and its precursors and rRNA. The probes of a target-specific probe set typically hybridize to adjacent regions on the target nucleic acid sequence such that, under appropriate conditions, they can be ligated together to form a ligation product. The skilled artisan will appreciate that the term “target nucleic acid sequence” encompasses more than one of the same species of sequences, and in certain embodiments, encompasses more than one species of sequences.

The term “quantitating,” when used in reference to an amplification product, refers to determining the quantity or amount of a particular detectable sequence that is representative of the target nucleic acid sequence in the sample. For example, but without limitation, measuring the fluorescent intensity of the reporter group detected at a specific address on a microarray or at the laser detection source of a capillary electrophoresis apparatus. The intensity or quantity of the detected reporter group is typically related to the amount of amplification product. The amount of amplification product generated correlates with the amount of target nucleic acid sequence present prior to ligation and amplification, and thus, may indicate the level of expression for a particular gene.

The term “amplification product” as used herein refers to the product of an amplification reaction including, but not limited to, primer extension, the polymerase chain reaction, RNA transcription, and the like. Thus, exemplary amplification products may comprise at least one of primer extension products, PCR amplicons, RNA transcription products, and the like.

Exemplary Reagents

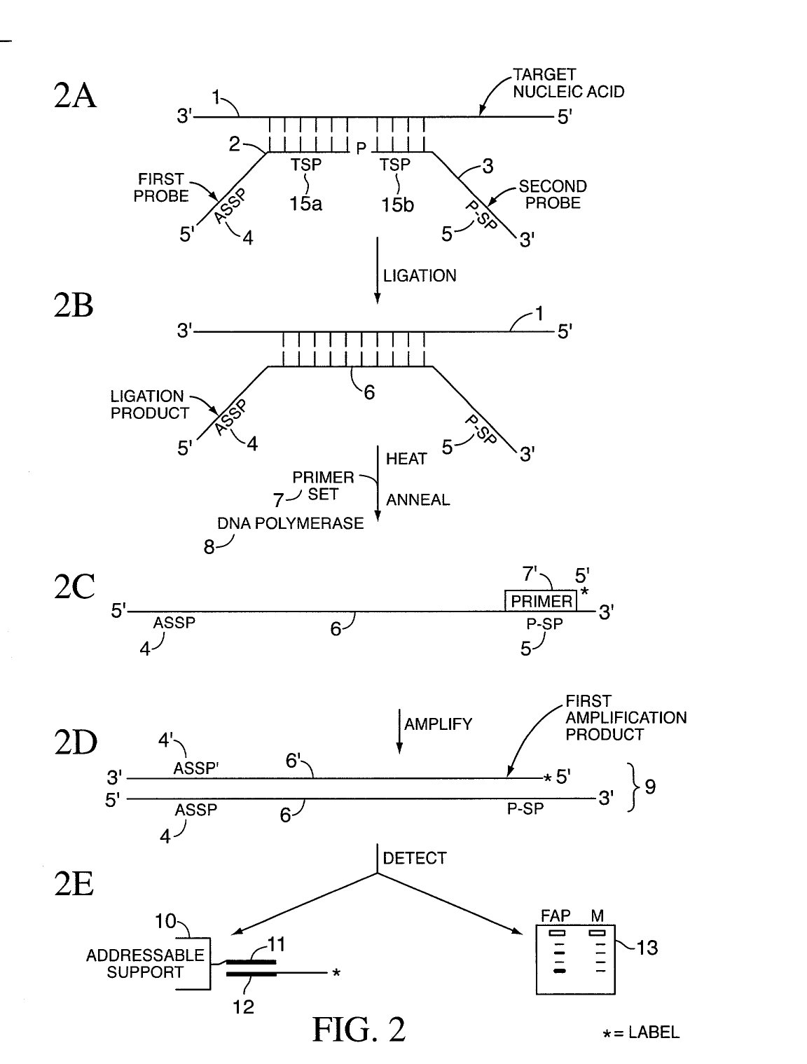

Probes, according to the present invention, are oligonucleotides that comprise a target-specific portion that is designed to hybridize in a sequence-specific manner with a complementary region on a specific target nucleic acid template (see, e.g., probes 2 and 3 in FIG. 2). A probe may further comprise a primer-specific portion, an addressable support-specific portion, all or part of a promoter or its complement, or a combination of these additional components. In certain embodiments, any of the probe's components may overlap any other probe component(s). For example, but without limitation, the target-specific portion may overlap the primer-specific portion, the promoter or its complement, or both. Also, without limitation, the addressable support-specific portion may overlap with the target-specific portion or the primer specific-portion, or both.

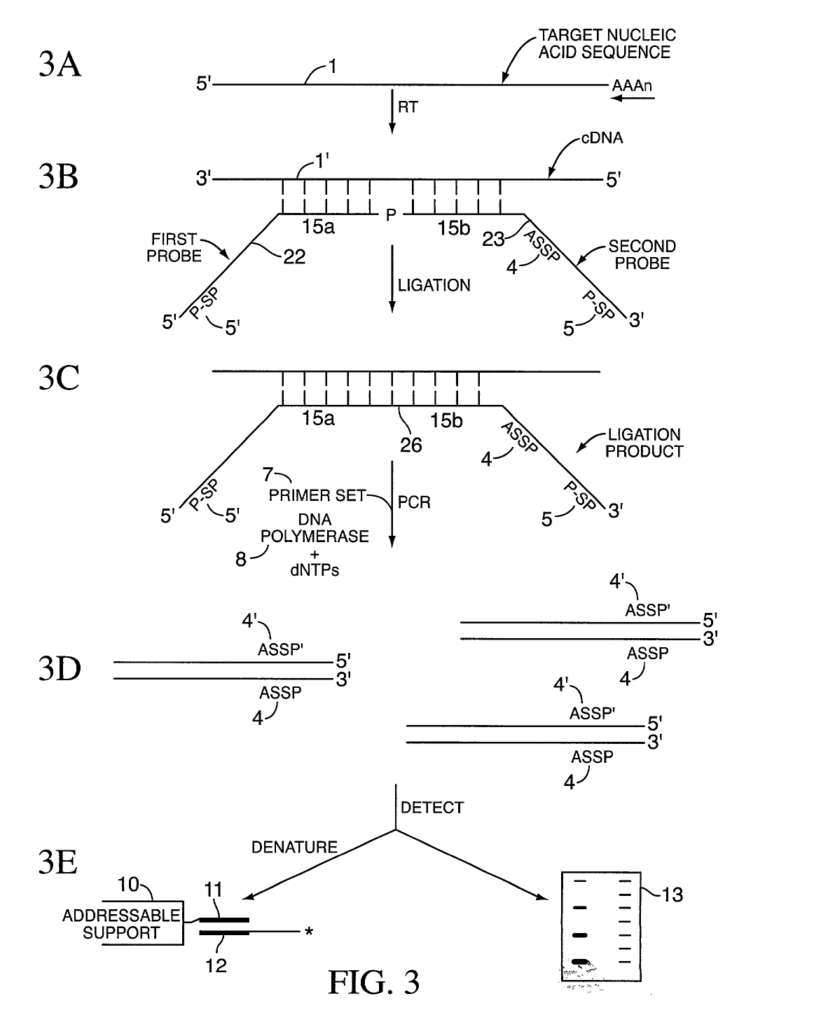

In certain embodiments, at least one probe of a probe set comprises the addressable support-specific portion located between the target-specific portion and the primer-specific portion (see, e.g., probe 23 in FIG. 3). The probe's addressable support-specific portion may comprise a sequence that is the same as, or complementary to, a portion of a capture oligonucleotide sequence located on an addressable support or a bridging oligonucleotide. Alternatively, the probe's addressable support-specific portion may comprise a mobility modifier that allows detection of the ligation or amplification products based on their location at a particular mobility address due to a mobility detection process, such as, but without limitation, electrophoresis. In one variation, each addressable support-specific portion is complementary to a particular mobility-modifier comprising a tag complement for selectively binding to the addressable support-specific portion of the amplification product, and a tail for effecting a particular mobility in a mobility-dependent analysis technique, e.g., electrophoresis, see, e.g., U.S. patent application Ser. No. 09/522,640, filed Mar. 15, 1999. In certain embodiments, the probe's addressable support-specific portion is not complementary with other target, probe, or primer sequences.

The sequence-specific portions of the probes are of sufficient length to permit specific annealing to complementary sequences in primers and targets. In certain embodiments, the length of the addressable support-specific portions and target-specific portion are 12 to 35 nucleotides. Detailed descriptions of probe design that provide for sequence-specific annealing can be found, among other places, in Diffenbach and Dveksler, PCR Primer, A Laboratory Manual, Cold Spring Harbor Press, 1995, and Kwok et al., Nucl. Acid Res. 18:999-1005 (1990).

A probe set according to the present invention comprises at least one first probe and at least one second probe that adjacently hybridize to the same target nucleic acid sequence. According to certain embodiments of the invention, a target-specific probe set is designed so that the target-specific portion of the first probe will hybridize with the downstream target region (see, e.g., probe 2 in FIG. 2) and the target-specific portion of the second probe will hybridize with the upstream target region (see, e.g., probe 3 in FIG. 2). The sequence-specific portions of the probes are of sufficient length to permit specific annealing with complementary sequences in targets and primers, as appropriate. In certain embodiments of the invention, both the at least one first probe and the at least one second probe in a probe set further comprise at least addressable support-specific portion. In certain embodiments, none of the addressable support-specific portions used in a particular reaction are complementary with any other portion in that reaction.

Under appropriate conditions, adjacently hybridized probes may be ligated together to form a ligation product, provided that they comprise appropriate reactive groups, for example, without limitation, a free 3′-hydroxyl or 5′-phosphate group. Some probe sets may comprise more than one first probe or more than one second probe.

Primers according to the present invention refer to oligonucleotides that are designed to hybridize with the primer-specific portion of probes, ligation products, or amplification products in a sequence-specific manner, and serve as primers for amplification reactions. A primer set according to the present invention comprises at least one primer capable of hybridizing with the primer-specific portion of at least one probe of a target-specific probe set. In certain embodiments, a primer set comprises at least one first primer and at least one second primer, wherein the at least one first primer specifically hybridizes with one probe of a target-specific probe set and the at least one second primer of the primer set specifically hybridizes with the other probe of the same target-specific probe set. In certain embodiments, at least one primer of a primer set further comprises all or part of a promoter sequence or its complement. In certain embodiments, the first and second primers of a primer set have different hybridization temperatures, to permit temperature-based asymmetric PCR reactions. The skilled artisan will appreciate that while the probes and primers of the invention may be described in the singular form, a plurality of probes or primers may be encompassed by the singular term, as will be apparent from the context. Thus, for example, in certain embodiments, a probe set typically comprises a plurality of first probes and a plurality of second probes.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

In embodiments that employ a promoter sequence, the promoter sequence or its complement will be of sufficient length to permit an appropriate polymerase to interact with it. Detailed descriptions of sequences that are sufficiently long for polymerase interaction can be found in, among other places, Sambrook and Russell.

According to certain embodiments, a primer set of the present invention comprises at least one second primer. The second primer in that primer set is designed to hybridize with a 3′ primer-specific portion of a ligation or amplification product in a sequence-specific manner (see, e.g., FIG. 2C). In certain embodiments, the primer set further comprises at least one first primer. The first primer of a primer set is designed to hybridize with the complement of the 5′ primer-specific portion of that same ligation or amplification product in a sequence-specific manner. In certain embodiments, at least one primer of the primer set comprises a promoter sequence or its complement or a portion of a promoter sequence or its complement. For a discussion of primers comprising promoter sequences, see Sambrook and Russell. In certain embodiments, at least one primer of the primer set further comprises a reporter group. In certain embodiments, reporter groups are fluorescent dyes attached to a nucleotide(s) in the primer (see, e.g., L. Kricka, Nonisotopic DNA Probe Techniques, Academic Press, San Diego, Calif. (1992)). In certain embodiments, the reporter group is attached to the primer in such a way as to not to interfere with sequence-specific hybridization or amplification.

According to certain embodiments, some probe sets may comprise more than one first probe or more than one second probe to allow sequence discrimination between target sequences that differ by one or more nucleotides (see, e.g., FIG. 7).

According to certain embodiments of the invention, a target-specific probe set is designed so that the target-specific portion of the first probe will hybridize with the downstream target region (see, e.g., probe A in FIG. 6) and the target-specific portion of the second probe will hybridize with the upstream target region (see, e.g., probe Z in FIG. 6). A nucleotide base complementary to the pivotal nucleotide, the “pivotal complement,” is present on the proximal end of either the first probe or the second probe of the target-specific probe set (see, e.g., 3′ end of A in FIG. 6).

When the first and second probes of the probe set are hybridized to the appropriate upstream and downstream target regions, and the pivotal complement is base-paired with the pivotal nucleotide on the target sequence, the hybridized first and second probes may be ligated together to form a ligation product (see, e.g., FIG. 7(b)-(c)). A mismatched base at the pivotal nucleotide, however, interferes with ligation, even if both probes are otherwise fully hybridized to their respective target regions. Thus, highly related sequences that differ by as little as a single nucleotide can be distinguished.

For example, according to certain embodiments, one can distinguish the two potential alleles in a biallelic locus as follows. One can combine a probe set comprising two first probes, differing in their primer-specific portions and their pivotal complement (see, e.g., probes A and B in FIG. 7(a)), one second probe (see, e.g., probe Z in FIG. 7(a)), and the sample containing the target. All three probes will hybridize with the target sequence under appropriate conditions (see, e.g., FIG. 7(b)). Only the first probe with the hybridized pivotal complement, however, will be ligated with the hybridized second probe (see, e.g., FIG. 7(c)). Thus, if only one allele is present in the sample, only one ligation product for that target will be generated (see, e.g., ligation product A-Z in FIG. 7(d)). Both ligation products would be formed in a sample from a heterozygous individual.

Further, in certain embodiments, probe sets do not comprise a pivotal complement at the terminus of the first or the second probe. Rather, the target nucleotide or nucleotides to be detected are located within either the 5′ or 3′ target region. Probes with target-specific portions that are fully complementary with their respective target regions will hybridize under high stringency conditions. Probes with one or more mismatched bases in the target-specific portion, by contrast, will not hybridize to their respective target region. Both the first probe and the second probe must be hybridized to the target for a ligation product to be generated. The nucleotides to be detected may be both pivotal or internal.

In certain embodiments, the first probes and second probes in a probe set are designed with similar melting temperatures (Tₘ). Where a probe includes a pivotal complement, preferably, the Tₘ for the probe(s) comprising the pivotal complement(s) of the target pivotal nucleotide sought will be approximately 4-6° C. lower than the other probe(s) that do not contain the pivotal complement in the probe set. The probe comprising the pivotal complement(s) will also preferably be designed with a Tₘ near the ligation temperature. Thus, a probe with a mismatched nucleotide will more readily dissociate from the target at the ligation temperature. The ligation temperature, therefore, provides another way to discriminate between, for example, multiple potential alleles in the target.

A “universal primer” is capable of hybridizing to the primer-specific portion of more than one species of probe, ligation product, or amplification product, as appropriate. A “universal primer set” comprises a first primer and a second primer that hybridize with a plurality of species of probes, ligation products, or amplification products, as appropriate. In certain embodiments, the universal primer or the universal primer set hybridizes with all or most of the probes, ligation products, or amplification products in a reaction, as appropriate. When universal primer sets are used in certain amplification reactions, such as, but not limited to, PCR, quantitative results may be obtained for a broad range of template concentrations.

A ligation agent according to the present invention may comprise any number of enzymatic or chemical (i.e., non-enzymatic) agents. For example, ligase is an enzymatic ligation agent that, under appropriate conditions, forms phosphodiester bonds between the 3′—OH and the 5′-phosphate of adjacent nucleotides in DNA or RNA molecules, or hybrids. Temperature sensitive ligases, include, but are not limited to, bacteriophage T4 ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eucaryotic, or archael organisms. Certain RNA ligases may also be employed in the methods of the invention. In certain embodiments, the ligation agent is an “activating” or reducing agent.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

A support or addressable support according to the present invention comprises a support such as a microarray, a microtiter plate, a membrane, beads, including, without limitation, coated or uncoated particles comprising magnetic and paramagnetic material, polyacrylamide, polysaccharide, plastic, and the like, that further comprise bound or immobilized spatially addressable oligonucleotide capture sequence(s), specific ligands, or the like. In certain embodiments, the addressable support-specific portion of amplification products or portions of the amplification products bind directly to the spatially addressable oligonucleotide capture sequence(s). In other embodiments, the addressable support-specific portion of the amplification products or portions of the amplification products bind indirectly to the support via bridging oligonucleotides. These bridging oligonucleotides are capable of hybridizing with both the spatially addressable oligonucleotide capture sequence and the addressable support-specific portion of the amplification product or its complement, or of a portion of the amplification product or its complement. Thus the bridging oligonucleotides serve as an intermediate between the capture sequence and the amplification product or portion of the amplification product.

In certain embodiments, a polymerase is used. In certain embodiments, the polymerase may comprise at least one thermostable polymerase, including, but not limited to, Taq, Pfu, Vent, Deep Vent, Pwo, UlTma, and Tth polymerase and enzymatically active mutants and variants thereof. Descriptions of these polymerases may be found, among other places, at the world wide web URL: the-scientist.library.upenn.edu/yr1998/jan/profile 1_{—}980105. html.

Addressable supports may have a wide variety of geometrys and configurations, and may be fabricated using any one of a number of different known fabrication techniques. Exemplary fabrication techniques include, but are not limited to, in situ synthesis techniques, e.g., Southern, U.S. Pat. No. 5,436,327 and related patents; light-directed in situ synthesis techniques, e.g., Fodor et al., U.S. Pat. No. 5,744,305 and related patents; robotic spotting techniques, e.g., Cheung et al., Nature Genetics, 21: 15-19 (1999), Brown et al., U.S. Pat. No. 5,807,522, Cantor, U.S. Pat. No. 5,631,134, or Drmanac, U.S. Pat. No. 6,025,136; or arrays of beads having oligonucleotides attached thereto, e.g., Walt, U.S. Pat. No. 6,023,540. Methods used to perform the hybridization process used with the supports are well known and will vary depending upon the nature of the support bound capture nucleic acid and the nucleic acid in solution, e.g., Bowtell, Nature Genetics, 21: 25-32 (1999); Brown and Botstein, Nature Genetics, 21: 33-37 (1999).

The skilled artisan will appreciate that the complement of the disclosed probe, target, and primer sequences, or combinations thereof, may be employed in the methods of invention. For example, without limitation, a genomic DNA sample comprises both the target sequence and its complement. Thus when a genomic sample is denatured, both the target sequence and its complement are present in the sample as single-stranded sequences. The probes described herein will specifically hybridize to the appropriate sequence, either the target or its complement.

Exemplary Methods

A target nucleic acid sequence for use with the present invention may be derived from any living, or once living, organism, including but not limited to prokaryote, eukaryote, plant, animal, and virus. The target nucleic acid sequence may originate from a nucleus of a cell, e.g., genomic DNA, or may be extranuclear nucleic acid, e.g., plasmid, mitochondrial nucleic acid, various RNAs, and the like. In certain embodiments, if the sequence from the organism is RNA, it may be reverse-transcribed into a cDNA target nucleic acid sequence. Furthermore, in certain embodiments, the target nucleic acid sequence may be present in a double stranded or single stranded form.

A variety of methods are available for obtaining a target nucleic acid sequence for use with the compositions and methods of the present invention. When the target nucleic acid sequence is obtained through isolation from a biological matrix, certain isolation techniques include (1) organic extraction followed by ethanol precipitation, e.g., using a phenol/chloroform organic reagent (e.g., Ausubel et al., Volume 1, Chapter 2, Section I), preferably using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)), such precipitation methods being typically referred to as “salting-out” methods. In certain embodiments, each of the above isolation methods is preceded by an enzyme digestion step to help eliminate unwanted protein from the sample, e.g., digestion with proteinase K, or other like proteases. See, e.g., U.S. patent application Ser. No. 09/724,613.

Ligation according to the present invention comprises any enzymatic or chemical process wherein an internucleotide linkage is formed between the opposing ends of nucleic acid sequences that are adjacently hybridized to a template. Additionally, the opposing ends of the annealed nucleic acid sequences are suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, T7 DNA ligase, Thermus thermophilus (Tth) ligase, Thermus aquaticus (Taq) ligase, or Pyrococcus furiosus (Pfu) ligase. Other internucleotide linkages include, without limitation, covalent bond formation between appropriate reactive groups such as between an α-haloacyl group and a phosphothioate group to form a thiophosphorylacetylamino group; and between a phosphorothioate, a tosylate, or iodide group to form a 5′-phosphorothioester or pyrophosphate linkages.

Chemical ligation may, under appropriate conditions, occur spontaneously such as by autoligation. Alternatively, “activating” or reducing agents may be used. Examples of activating agents and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light. Nonenzymatic ligation according to certain embodiments may utilize specific reactive groups on the respective 3′ and 5′ ends of the aligned probes.

Ligation generally comprises at least one cycle of ligation, i.e., the sequential procedures of: hybridizing the target-specific portions of a first probe and a second probe, that are suitable for ligation, to their respective complementary regions on a target nucleic acid sequence; ligating the 3′ end of the first probe with the 5′ end of the second probe to form a ligation product; and denaturing the nucleic acid duplex to separate the ligation product from the target nucleic acid sequence. The cycle may or may not be repeated. For example, without limitation, by thermocycling the ligation reaction to linearly increase the amount of ligation product.

Also within the scope of the invention are ligation techniques such as gap-filling ligation, including, without limitation, gap-filling OLA and LCR, bridging oligonucleotide ligation, and correction ligation. Descriptions of these techniques can be found, among other places, in U.S. Pat. No. 5,185,243, published European Patent Applications EP 320308 and EP 439182, and published PCT Patent Application WO 90/01069.

When used in the context of the present invention, “suitable for ligation” refers to at least one first probe and at least one second probe, each comprising an appropriate reactive group. Exemplary reactive groups include, but are not limited to, a free hydroxyl group on the 3′ end of the first probe and a free phosphate group on the 5′ end of the second probe, phosphorothioate and tosylate or iodide, esters and hydrazide, RC(O)S⁻, haloalkyl, RCH₂S and α-haloacyl, thiophosphoryl and bromoacetamide groups, and S-pivaloyloxymethyl-4-thiothymidine. Additionally, in certain embodiments, the first and second probes are hybridized to the target such that the 3′ end of the first probe and the 5′ end of the second probe are immediately adjacent to allow ligation.

Purifying the ligation product according to the present invention comprises any process that removes at least some unligated probes, target nucleic acid sequences, enzymes or accessory agents from the ligation reaction mixture following at least one cycle of ligation. Such processes include, but are not limited to, molecular weightsize exclusion processes, e.g., gel filtration chromatography or dialysis, sequence-specific hybridization-based pullout methods, affinity capture techniques, precipitation, adsorption, or other nucleic acid purification techniques. The skilled artisan will appreciate that purifying the ligation product prior to amplification reduces the quantity of primers needed to amplify the ligation product, thus reducing the cost of detecting a target sequence. Also, purifying the ligation product prior to amplification decreases possible side reactions during amplification and reduces competition from unligated probes during hybridization.

Hybridization-based pullout (HBP) according to the present invention comprises a process wherein a nucleotide sequence complementary to at least a portion of one probe, for example, the primer-specific portion, is bound or immobilized to a solid or particulate pullout support (see, e.g., U.S. patent application Ser. No. 08/873,437 to O'Neill et al., filed Jun. 12, 1997). The ligation reaction mixture (comprising the ligation product, target sequences, and unligated probes) is exposed to the pullout support. The ligation product, under appropriate conditions, hybridizes with the support-bound sequences. The unbound components of the ligation reaction mixture are removed, purifying the ligation products from those ligation reaction mixture components that do not contain sequences complementary to the sequence on the pullout support. One subsequently removes the purified ligation products from the support and combines it with at least one primer set to form a first amplification reaction mixture. The skilled artisan will appreciate that additional cycles of HBP using different complementary sequences on the pullout support will remove all or substantially all of the unligated probes, further purifying the ligation product.

Amplification according to the present invention encompasses a broad range of techniques for amplifying nucleic acid sequences, either linearly or exponentially. Exemplary amplification techniques include, but are not limited to, PCR or any other method employing a primer extension step, and transcription or any other method of generating at least one RNA transcription product. Other nonlimiting examples of amplification are ligase detection reaction (LDR), and ligase chain reaction (LCR). Amplification methods may comprise thermal-cycling or may be performed isothermally. The term “amplification product” includes first amplification products, second amplification products, primer extension products, and RNA transcription products, unless otherwise apparent from the context.

In certain embodiments, amplification methods comprise at least one cycle of amplification, for example, but not limited to, the sequential procedures of: hybridizing primers to primer-specific portions of the ligation product, first amplification product, or second amplification product; synthesizing a strand of nucleotides in a template-dependent manner using a polymerase; and denaturing the newly-formed nucleic acid duplex to separate the strands. The cycle may or may not be repeated. In certain embodiments, amplification methods comprise at least one cycle of amplification, for example, but not limited to, the sequential procedures of: interaction of a polymerase with a promoter; synthesizing a strand of nucleotides in a template-dependent manner using a polymerase; and denaturing the newly-formed nucleic acid duplex to separate the strands. The cycle may or may not be repeated.

Primer extension according to the present invention is an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand. Detailed descriptions of primer extension according to certain embodiments can be found, among other places in Sambrook et al., Sambrook and Russell, and Ausbel et al.

Transcription according to the present invention is an amplification process comprising an RNA polymerase interacting with a promoter on a single- or double-stranded template and generating a RNA polymer in a 5′ to 3′ direction. In certain embodiments, the transcription reaction mixture further comprises transcription factors. RNA polymerases, including but not limited to T3, T7, and SP6 polymerases, according to certain embodiments, can interact with either single-stranded or double-stranded promoters. Detailed descriptions of transcription according to certain embodiments can be found, among other places in Sambrook et al., Sambrook and Russell, and Ausbel et al.

Certain embodiments of amplification may employ multiplex PCR, in which multiple target sequences are simultaneously amplified using multiple sets of primers (see, e.g., H. Geada et al., Forensic Sci. Int. 108:31-37 (2000) and D. G. Wang et al., Science 280:1077-82 (1998)).

According to the present invention, analyzing or detecting comprises a process for identifying the presence or absence of a particular amplification product or a portion of an amplification product (i) at a specific address on an addressable support or (ii) occupying a particular mobility address. In certain embodiments, the process includes identifying the presence or absence of a particular amplification product or a portion of an amplification product during or as the result of a separation technique, for example, but not limited to, a mobility dependent analytical technique. In certain embodiments, when the addressable support-specific portion of an amplification product, or its complement, specifically hybridizes to the capture sequence on the addressable support, the hybridized sequence can be detected provided that a reporter group is present. Typically, the reporter group provides an emission that is detectable or otherwise identifiable in the detection step. The type of detection process used will depend on the nature of the reporter group to be detected. In certain embodiments, the detection process comprises laser-excited fluorescent detection of a fluorescent reporter group.

In certain embodiments, microarrays may be used for detection. (See, for example, Barany et al., PCT Publication No. WO 97/31256, published Aug. 28, 1997). Descriptions of these conventional amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Ausbel et al.

Quantitating, according to the present invention comprises determining the amount of the amplification product or portion of the amplification product (including primer extension products and transcription products). In certain embodiments, one quantitates by measuring the intensity of the reporter group present. The amount of a specific amplification product provides an indication of the amount of the corresponding target nucleic acid sequence that is initially present. In certain embodiments, when the gene expression levels for several target nucleic acid sequences for a sample are known, a gene expression profile for that sample can be compiled and compared with other samples. For example, but without limitation, samples may be obtained from two aliquots of cells from the same cell population, wherein one aliquot was grown in the presence of a chemical compound or drug and the other aliquot was not. By comparing the gene expression profiles for cells grown in the presence of drug with those grown in the absence of drug, one may be able to determine the drug effect on the expression of particular target genes.

Generating a single-stranded sequence for hybridization according to the present invention comprises a process for creating single-stranded nucleic acid molecules, or regions within molecules, to facilitate direct or indirect hybridization with an addressable support. Processes for generating single-stranded sequence for hybridization include, without limitation, denaturing double-stranded nucleic acid molecules by heating or using chemical denaturants; limited or complete exonuclease digestion of double-stranded nucleic acid molecules; asymmetric PCR; asynchronous PCR; and primer extension. Detailed descriptions of such processes can be found, among other places, in Ausbel et al., Sambrook and Russell, and Sambrook et al.

Asymmetric PCR according to the present invention comprises an amplification reaction mixture comprising (i) at least one primer set in which there is an excess of one primer (relative to the other primer in the primer set); (ii) at least one primer set that comprises only a first primer or only a second primer; (iii) at least one primer set that, during given amplification conditions, comprises a primer that results in amplification of one strand and comprises another primer that is disabled; or (iv) at least one primer set that meets the description of both (i) and (iii) above. Consequently, when the ligation product is amplified, an excess of one strand of the amplification product (relative to its complement) is generated. In certain embodiments, the single-stranded amplification product may then be hybridized directly with the support-bound capture oligonucleotides. In certain embodiments, the single-stranded amplification product may be separated by molecular weight, length, or mobility.

In certain embodiments, one may use at least one primer set wherein the Tm50 of one of the primers is higher than the Tm50 of the other primer. Such embodiments have been called asynchronous PCR (A-PCR). See, e.g., U.S. patent application Ser. No. 09/875,211, filed Jun. 5, 2001. In certain embodiments, the Tm50 of the first primer is at least 8-15° C. different from the Tm50 of the second primer. In certain embodiments, the Tm50 of the first primer is at least 10-15° C. different from the Tm50 of the second primer. In certain embodiments, the Tm50 of the first primer is at least 10-120 C. different from the Tm50 of the second primer. In certain embodiments of A-PCR, in addition to the difference in Tm50 of the primers in a primer set, there is also an excess of one primer relative to the other primer in the primer set. In certain embodiments, there is a five to twenty-fold excess of one primer relative to the other primer in the primer set. In certain embodiments of A-PCR, the primer concentration is at least 50 mM.

In A-PCR according to certain embodiments, one may use conventional PCR in the first cycles such that both primers anneal and both strands are amplified. By raising the temperature in subsequent cycles, however, one may disable the primer with the lower Tm such that only one strand is amplified. Thus, the subsequent cycles of A-PCR in which the primer with the lower Tm is disabled result in asymmetric amplification. Consequently, when the ligation product is amplified, an excess of one strand of the amplification product (relative to its complement) is generated. In certain embodiments, the single-stranded amplification product may then be hybridized directly with the support-bound capture oligonucleotides. In certain embodiments, the single-stranded amplification product may be separated by molecular weight, length, or mobility.

According to certain embodiments of A-PCR, the level of amplification can be controlled by changing the number of cycles during the first phase of conventional PCR cycling. In such embodiments, by changing the number of initial conventional cycles, one may vary the amount of the double strands that are subjected to the subsequent cycles of PCR at the higher temperature in which the primer with the lower Tm is disabled.

In certain embodiments, an A-PCR protocol may comprise use of a pair of primers, each of which has a concentration of at least 50 mM. In certain embodiments, conventional PCR, in which both primers result in amplification, is performed for the first 20-30 cycles. In certain embodiments, after 20-30 cycles of conventional PCR, the annealing temperature increases to 66-70° C., and PCR is performed for 5 to 40 cycles at the higher annealing temperature. In such embodiments, the lower Tm primer is disabled during such 5 to 40 cycles at higher annealing temperature. In such embodiments, asymmetric amplification occurs during the second phase of PCR cycles at a higher annealing temperature.

Asymmetric reamplification according to the present invention comprises generating single-stranded amplification product in a second amplification process. In certain embodiments, the double-stranded amplification product of a first amplification process serves as the amplification target in the asymmetric reamplification process. In certain embodiments, one may achieve asymmetric reamplification using asynchronous PCR in which initial cycles of PCR conventionally amplify two strands and subsequent cycles are performed at a higher annealing temperature that disables one of the primers of a primer set as discussed above. In certain embodiments, the second amplification reaction mixture comprises at least one primer set which comprises the at least one first primer, or the at least one second primer of a primer set, but typically not both. The skilled artisan understands that asymmetric reamplification will also eventually occur if the primers in the primer set are not present in an equimolar ratio. In certain asymmetric reamplification methods, typically only single-stranded amplicons are generated since the second amplification reaction composition comprises only first or second primers from each primer set or a non-equimolar ratio of first and second primers from a primer set.

In certain embodiments, the primer in the second amplification reaction mixture comprises a reporter group so that the single-stranded second amplification product is labeled and may be detected when hybridized to the capture or bridging oligonucleotides on the addressable support or when occupying a particular mobility address.

In certain embodiments, additional polymerase may also be a component of the second amplification reaction mixture. In certain embodiments, there may be sufficient residual polymerase from the first amplification mixture to synthesize the second amplification product.

Separating by molecular weight or length or mobility according to the present invention is used in the broad sense. Any method that allows a mixture of two or more nucleic acid sequences to be distinguished based on the mobility, molecular weight, or nucleotide length of a particular sequence is within the scope of the invention. Exemplary procedures include, without limitation, electrophoresis, such as gel or capillary electrophoresis, HPLC, mass spectroscopy including MALDI-TOF, and gel filtration.

In certain embodiments, one may quantitate the amount of mRNA encoding a particular protein within a cell to determine a particular condition of an individual. For example, the protein insulin, among other things, regulates the level of blood glucose. The amount of insulin that is produced in an individual can determine whether that individual is healthy or not. Insulin deficiency results in diabetes, a potentially fatal disease. Diabetic individuals typically have low levels of insulin mRNA and thus will produce low levels of insulin, while healthy individuals typically have higher levels of insulin mRNA and produce normal levels of insulin.

Another human disease typically due to abnormally low gene expression is Tay-Sachs disease. Children with Tay-Sachs disease lack, or are deficient in, a protein(s) required for sphingolipid breakdown. These children, therefore, have abnormally high levels of sphingolipids causing nervous system disorders that may result in death.

In certain embodiments, it is useful to identify and detect additional genetic-based diseases/disorders that are caused by gene over- or under-expression. Additionally, cancer and certain other known diseases or disorders can be detected by, or are related to, the over- or under-expression of certain genes. For example, men with prostate cancer typically produce abnormally high levels of prostate specific antigen (PSA); and proteins from tumor suppressor genes are believed to play critical roles in the development of many types of cancer.

Using nucleic acid technology, in certain embodiments, minute amounts of a biological sample can typically provide sufficient material to simultaneously test for many different diseases, disorders, and predispositions. Additionally, there are numerous other situations where it would be desirable to quantify the amount of specific target nucleic acids, particularly mRNA, in a cell or organism, a process sometimes referred to as “gene expression profiling.” When the quantity of a particular target nucleic acid within, for example, a specific cell-type or tissue, or an individual is known, in certain cases one may start to compile a gene expression profile for that cell-type, tissue, or individual. Comparing an individual's gene expression profile with known expression profiles may allow the diagnosis of certain diseases or disorders in certain cases. Predispositions or the susceptibility to developing certain diseases or disorders in the future may also be identified by evaluating gene expression profiles in certain cases. Gene expression profile analysis may also be useful for, among other things, genetic counseling and forensic testing in certain cases.

Certain Exemplary Embodiments of Determining Target Sequences

The present invention is directed to methods, reagents, and kits for quantitating target nucleic acid sequences in a sample, using coupled ligation and amplification reactions to generate amplification products, including, but not limited to, first amplification products, second amplification products, primer extension products, and RNA transcription products. The amplification products are analyzed and quantitated using the addressable support-specific portion of the amplification product. For example, but not limited to, (i) addressable support-specific portions of the amplification products hybridized directly or indirectly to an addressable support, or (ii) amplification products or portions of amplification products present at particular mobility addresses, for example, during or after a separation process.

In certain embodiments, one or more nucleic acid species (A) are subjected to coupled ligation (B1-B3) and amplification (C1-C3) reactions, either directly or via an intermediate, such as a cDNA target generated from an mRNA by reverse transcription (A1). In certain embodiments, one or more target nucleic acid species may be subjected directly to at least one ligation reactions (e.g., B1, B3), coupled to at least one amplification reaction, such as in vitro transcription (C1), asymmetric PCR (C3), primer extension, or PCR (C2), to generate at least one first amplification product, which may comprise either double-stranded molecules, single-stranded molecules (e.g., D), or both double- and single-stranded molecules. In certain embodiments, the initial nucleic acid comprises mRNA and a reverse transcription reaction may be performed to generate at least one cDNA (e.g., A1), followed by at least one ligation reaction (e.g., B2) coupled to at least one amplification reaction (e.g., C2). The first amplification products may be detected and quantified using for example array hybridization (E) or by a technique that distinguishes nucleic acids based on mobility, weight, or size. In certain embodiments, at least one first amplification product may be subjected to a second amplification reaction to generate a second amplification product, that is subsequently detected and quantitated. In certain embodiments, at least one first amplification product is subjected to enzymatic digestion to generate at least one single-stranded digestion product, that is subsequently detected and quantitated. In certain embodiments, the amount of target nucleic acid sequence present after each reaction can be quantitated using conventional TaqMan assays (e.g., T1-T4).

In certain embodiments, for each target nucleic acid sequence to be detected, a probe set, comprising at least one first probe and at least one second probe, is combined with the sample to form a ligation reaction mixture. In certain embodiments, the ligation mixture may further comprise a ligation agent. In certain embodiments, the first and second probes in each probe set are suitable for ligation and are designed to hybridize to adjacent sequences that are present in the target nucleic acid sequence. When the target sequence is present in the sample, the first and second probes will, under appropriate conditions, hybridize to adjacent regions on the target nucleic acid sequence (see, e.g., probes 2 and 3 hybridized to target nucleic acid sequence 1 in FIG. 2A). In FIG. 2A, the target nucleic acid sequence (1) is depicted as hybridized with a first probe (2), for illustration purposes shown here as comprising an addressable support-specific portion (4) and a target-specific portion (15a), and a second probe (3) comprising a 3′ primer-specific portion (5), a target-specific portion (15b) and a free 5′ phosphate group (“P”) for ligation.

In certain embodiments, the adjacently hybridized probes may, under appropriate conditions, be ligated together to form a ligation product (see, e.g., ligation product 6 in FIG. 2B). FIG. 2B depicts the ligation product (6), generated from the ligation of the first probe (2) and the second probe (3). The ligation product (6) is shown comprising the addressable support-specific portion (4) and the 3′ primer-specific portion (5). In certain embodiments, when the duplex comprising the target nucleic acid sequence (1) and the ligation product (6) is denatured, for example, by heating, the ligation product (6) is released.

In certain embodiments, the ligation product 6 (in appropriate salts, buffers, and nucleotide triphosphates) is combined with at least one primer set 7 and a polymerase 8 to form a first amplification reaction mixture (see, e.g., FIGS. 2C-2D). In the first amplification cycle, the second primer 7′, comprising a sequence complementary to the 3′ primer-specific portion 5 of the ligation product 6, hybridizes with the ligation product 6 and is extended, in the presence of DNA polymerase and deoxynucleoside triphosphates (dNTPs), in a template-dependent fashion to create a double-stranded molecule 9 comprising the ligation product 6 and its complement 6′ (see, e.g., FIGS. 2C-D). In certain embodiments, the primer 7′ further comprises a reporter group, denoted by the symbol “*” in FIG. 2. The amplification product (9) comprises both the addressable support-specific portion (4) and the complement of the addressable support-specific portion (4′).

When the ligation product exists as a double-stranded molecule 9, in certain embodiments, subsequent amplification cycles may exponentially amplify this molecule, as shown in FIG. 3. In certain embodiments, the primers comprise reporter groups and the reporter group(s) of the first primers of the primer set are different from the reporter group(s) of the second primers. In other embodiments the primers of a primer set comprise the same reporter group(s). In yet other embodiments, either the first primer or the second primer, but not both, further comprise at least one reporter group. In certain embodiments, neither the first primer nor the second primer in a primer set comprises a reporter group. In certain embodiments, at least one primer further comprises all or part of a promoter or its complement. Certain embodiments further comprise a second amplification procedure.

In certain embodiments, following at least one amplification cycle, as shown in FIGS. 2 and 3, the addressable support-specific portions or complements thereof 12 of the amplification products or portions of the amplification products are specifically hybridized directly with capture oligonucleotides 11 on an addressable support 10 or indirectly via bridging oligonucleotides. The presence and amount of a particular target sequence in the sample is determined by detecting and quantitating a hybridized amplification product on the support 10. Alternatively, in certain embodiments, detection may comprise separation, provided that the addressable support-specific portion imparts a particular molecular weight, length, or mobility on the amplification product or a portion of the amplification product. The separation may be, for example, but not limited to electrophoresis, as depicted in FIG. 2E (13).

As shown in FIG. 3A, in certain embodiments, an mRNA is used to generate a cDNA copy 1′. The cDNA serves as a target nucleic acid sequence to which the first and second probes of the probe set hybridize (see FIG. 3B). The first probe 22 further comprises a 5′ primer-specific portion (5′) and a target-specific portion 15a and the second probe 23 comprises a target-specific portion 15b, an addressable support-specific portion 4, and a 3′ primer-specific portion (5). Under appropriate conditions, the adjacently hybridized probes can form a ligation product 26 comprising a 5′ primer-specific portion (5′), the target-specific portions 15a and 15b, the addressable support-specific portion 4, and the 3′ primer-specific portion (5) (see FIG. 3C).

When the duplex formed by the target nucleic acid sequence 1′ and the ligation product 26 is denatured, typically by heating, the ligation product is released. In the presence of the appropriate primer set and under appropriate conditions, the 3′ primer hybridizes with the 3′ primer-specific portion 5 of the ligation product 26. The 3′ primer is extended in the presence of DNA polymerase 8, generating a double-stranded product that comprises complement of the 5′ primer-specific portion of the ligation product (see FIG. 3D). The double-stranded primer-extension product is denatured and subjected to one or more cycles of the polymerase chain reaction (PCR) to generate first amplification products (see FIG. 3D). The first amplification products are then detected and quantitated (see FIG. 3E).

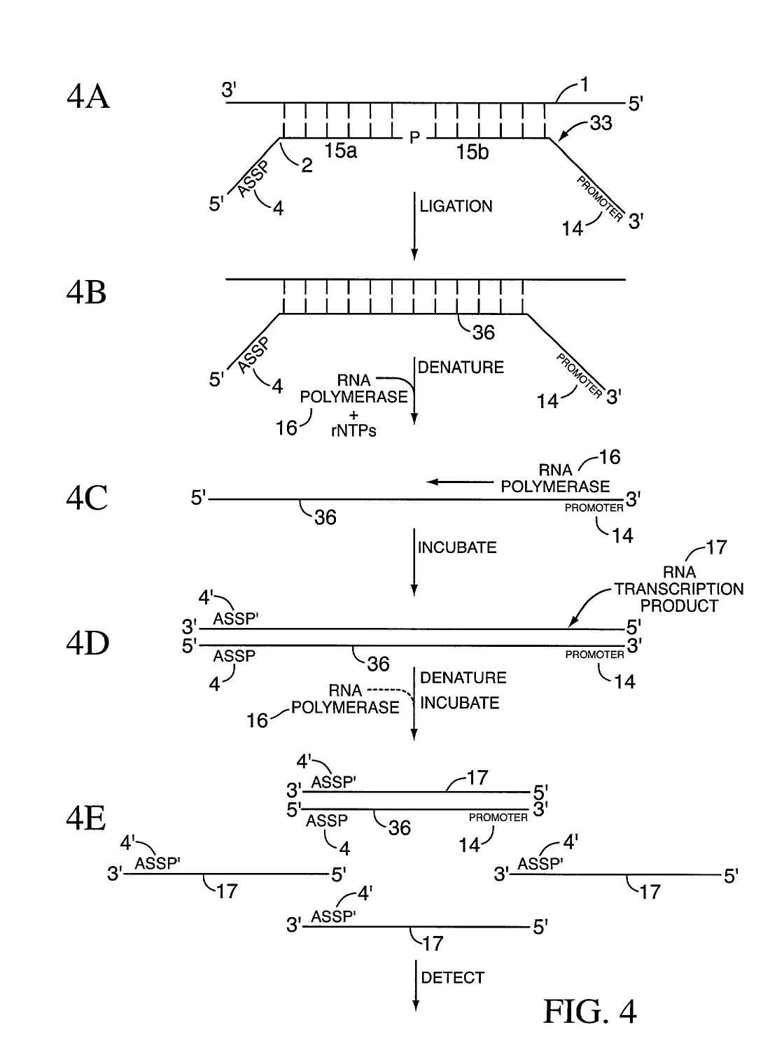

In certain embodiments as shown in FIG. 4A, the first probe 2, comprising an addressable support-specific portion 4, and the second probe 33, comprising a promoter 14, are shown hybridized with the target nucleic acid sequence 1. The adjacently hybridized probes are ligated together to form a duplex that contains the target nucleic acid sequence 1 and the ligation product 36 comprising an addressable support-specific portion 4 and a promoter 14, as shown in FIG. 4B. When the duplex is denatured, the ligation product is released. In the presence of an appropriate RNA polymerase 16, promoter interaction occurs, as shown in FIG. 4C, and under appropriate conditions one or more RNA transcription products 17 comprising the complement of the addressable support-specific portion 4′ is formed. Multiple RNA transcription products 17 can be generated from the ligation product, under appropriate conditions, as shown in FIG. 4E. The RNA transcription products are then detected and quantitated.

The skilled artisan will understand that some RNA polymerases typically form RNA transcription product(s) using a double-stranded transcription template, but not single-stranded transcription templates. Thus, when employing such RNA polymerases, a double-stranded version of the ligation product is typically generated before transcription occurs, as shown for example, in FIG. 4. The skilled artisan will also understand that it may be desirable to add RNA polymerase after some or all of the denaturation procedures.

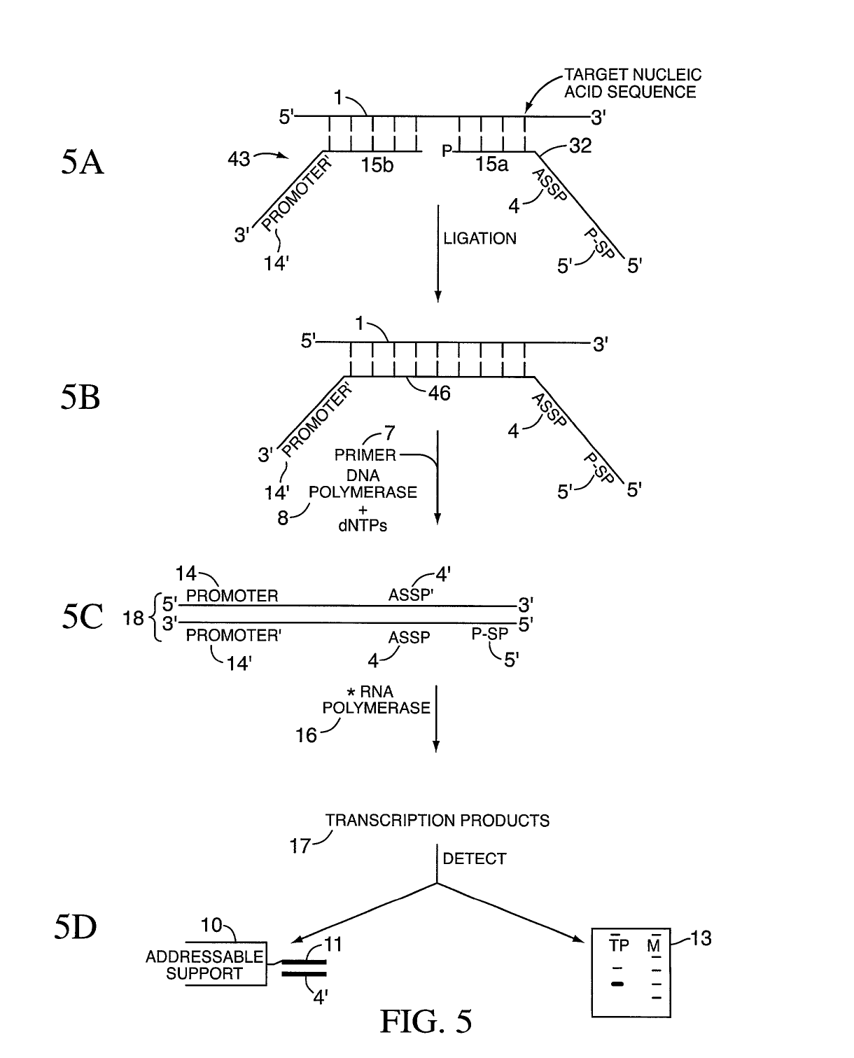

In certain embodiments as shown in FIG. 5A, the first probe 32, comprising a 5′ primer-specific portion 5′, an addressable support-specific portion 4, and a target-specific portion 15a, and the second probe 43, comprising a target-specific portion 15b and a complement of a promoter 14′, are shown hybridized with the target nucleic acid sequence 1. The adjacently hybridized probes are ligated together to form a duplex that contains the target nucleic acid sequence 1 and the ligation product 46 comprising an addressable support-specific portion 4 and the promoter complement 14′, as shown in FIG. 5B. When the duplex is denatured, the ligation product 46 is released. As shown in FIG. 5C, under appropriate conditions and in the presence of appropriate primers 7 and DNA polymerase 8, a double-stranded first amplification product 18 is generated, comprising the promoter 14 and its complement 14′, and the addressable support-specific portion 4 and its complement 4′. The first amplification product is transcribed under appropriate conditions and in the presence of RNA polymerase 16 to generate transcription products 17. The transcription products may be detected and quantitated by, for example hybridization of the complement of the addressable support-specific portion 4′ to appropriate capture oligonucleotides 11 on an addressable array 10 or a mobility-dependent analysis technique, such as, but not limited to, electrophoresis 13.

According to certain embodiments, the first and second probes in each probe set are designed to be complementary to the sequences immediately flanking the pivotal nucleotide of the target sequence (see, e.g., probes A, B, and Z in FIG. 8(a)). Either the at least one first probe or the at least one second probe of a probe set, but not both, will comprise the pivotal complement (see, e.g., probe A of FIG. 8(a)). When the target sequence is present in the sample, the first and second probes will hybridize, under appropriate conditions, to adjacent regions on the target (see, e.g., FIG. 8(b)). When the pivotal complement is base-paired in the presence of an appropriate ligation agent, two adjacently hybridized probes may be ligated together to form a ligation product (see, e.g., FIG. 8(c)).

The ligation reaction mixture (in the appropriate salts, buffers, and nucleotide triphosphates) is then combined with at least one primer set and a polymerase to form a first amplification reaction mixture (see, e.g., FIG. 8(d)). In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement (see, e.g., FIGS. 8(d)-(e)). When the ligation product exists as a double-stranded molecule, subsequent amplification cycles may exponentially amplify this molecule (see, e.g., FIGS. 8(d)-(h)). In FIG. 8, for example, primers PA* and PB* include different reporter groups. Thus, amplification products resulting from incorporation of these primers will include a reporter group specific for the particular pivotal nucleotide that is included in the original target sequence. Certain embodiments of the invention further comprise a second amplification procedure.

Following at least one amplification cycle, the addressable support-specific portions of the amplification products are specifically hybridized with capture oligonucleotides on an addressable support (see, e.g., FIGS. 8(i)-(j)). The presence of a particular target sequence in the sample is determined by detecting a hybridized amplification product on the support (see, e.g., FIG. 8(k)). As shown in FIG. 8, for example, according to certain embodiments, one can detect the presence of a particular pivotal nucleotide depending on the reporter group detected on the support.

In certain embodiments, the addressable support-specific portion of the amplification product may be single-stranded to optimize hybridization to an addressable support. In certain embodiments, a single-stranded amplification product is synthesized by, for example, without limitation, asymmetric PCR, primer extension, RNA polymerase (see, e.g., FIG. 4 and FIG. 5) or asymmetric reamplification.

In an exemplary embodiment of asymmetric PCR, the amplification reaction mixture is prepared with at least one primer set, wherein either the at least one first primer, or the at least one second primer, but not both, are added in excess. Thus, in certain embodiments, the excess primer to limiting primer ratio may be approximately 100:1, respectively. The ideal amounts of the primers according to certain embodiments may be determined empirically. In certain embodiments, amounts will range from about 0.2 to 1 pmol for the limiting primer, and from about 10 to 30 pmol for the primer in excess. Empirically, in certain embodiments, the concentration of one primer in the primer set is typically kept below 1 pmol per 100 μl of amplification reaction mixture.

Since both primers are initially present in substantial excess at the beginning of the PCR reaction in certain embodiments, both strands are exponentially amplified. In certain embodiments, prior to completing all of the cycles of amplification, however, the limiting primer is exhausted. During the subsequent cycles of amplification, only one strand is amplified, thus generating single-stranded amplification products.

For example, but without limitation, in certain embodiments, after approximately 40 to 45 cycles of amplification are performed, the amplification process is completed with a long extension step. The limiting primer is typically exhausted by the 25^{th} cycle of amplification. During subsequent cycles of amplification only one strand of the amplification product is produced due to the presence of only one primer of the primer set. At the completion of the amplification process the reaction mixture contains a substantial amount of single-stranded amplification product that can be hybridized directly with capture oligonucleotides on the addressable support.

In one exemplary asymmetric reamplification protocol, an air-dried first amplification mixture containing double-stranded amplification product, is resuspended in 30 μl of 0.1×TE buffer, pH 8.0. The second amplification reaction mixture is prepared by combining two microliters of the resuspended amplification product in a 0.2 ml MicroAmp reaction tube with 9 μl sterile filtered deionized water, 18 μl AmpliTaq Gold mix (PE Biosystems, Foster City, Calif.), and 20-40 pmol of either the at least one first primer or the at least one second primer suspended in 1 μl 1×TE buffer. Either the at least one first primer, the at least one second primer, or both are labeled.

The tubes are heated to 95° C. for 12 minutes, then cycled for ten cycles of (94° C. for 15 seconds, 60° C. for 15 seconds, and 72° C. for 30 seconds), followed by twenty-five cycles of (89° C. for 15 seconds, 53° C. for 15 seconds, and 72° C. for 30 seconds), and then 45 minutes at 60° C. The second amplification reaction mixture, containing single-stranded amplification product, is then cooled to 4° C.

Unincorporated PCR primers may be removed from the reaction mixture as follows. To each 30 μl amplification reaction mixture 0.34 μl of glycogen (10 mg/ml), 3.09 μl 3 M sodium acetate buffer, pH 5, and 20.6 μl absolute isopropanol are added. The tubes are mixed by vortexing and incubated at room temperature for ten minutes followed by centrifugation at 14,000 rpm for 10-15 minutes in a Beckman Model 18 microfuge.

Supernatants are removed from the labeled amplification product pellets. Each pellet is washed with 50 μl of 70% ethanol with vortexing. The washed amplification products are centrifuged at 14,000 rpm for 5 minutes in a Beckman Model 18 microfuge and the supernatant is removed. The pellets are washed again using 50 μl anhydrous ethanol, vortexed, and centrifuged at 14,000 rpm for 5 minutes, as before. The pellets are air-dried. The dried pellets may be stored at 4° C. prior to hybridization.

In other embodiments, a double-stranded amplification product is generated and subsequently converted into single-stranded sequences. Processes for converting double-stranded nucleic acid into single-stranded sequences include, without limitation, heat denaturation, chemical denaturation, and exonuclease digestion. Detailed protocols for synthesizing single-stranded nucleic acid molecules or converting double-stranded nucleic acid into single-stranded sequences can be found, among other places, in Ausbel et al., Sambrook et al., the Novagen Strandase™ product insert (Novagen, Madison, Wis.), and Sambrook and Russell.

The skilled artisan will appreciate, however, that when a single-stranded sequence is generated by denaturing a double-stranded sequence, the complementary single-stranded sequences may renature during the support hybridization process. Thus, when using such a denaturation process in certain embodiments, the number of single-stranded sequences available for hybridization with an addressable support may be decreased.

An exemplary nuclease digestion protocol is as follows. An air-dried first amplification product is resuspended in 10 μl sterile water. Eight microliters of the resuspended amplification product is combined with 1 μl Strandase buffer (Novagen, Madison, Wis.), and 1 μl exonuclease (5 units/μl) in a 0.2 ml MicroAmp reaction tube. The tube is incubated for 20 minutes at 37° C. and the reaction stopped by heating for an additional 10 minutes at 75° C. In certain embodiments, the nuclease digestion composition will contain single-stranded or substantially single-stranded first amplification products suitable for hybridization with an addressable support. In certain embodiments, the single-stranded amplification products may be detected and quantitated based on their molecular weight, length, or mobility.

The skilled artisan will understand that certain exonucleases, for example, but without limitation, λ exonuclease, digest one strand of a double-stranded molecule from a 5′ phosphorylated end. Thus the first amplification product typically serves as a suitable template for nuclease digestion. Suitable templates can be generated during the first amplification process using phosphorylated primers as appropriate. That is, the strand of the amplification product that is to be hybridized with the support will not comprise a primer that is phosphorylated at the 5′-end, while the complementary strand will comprise a 5′ phosphorylated primer. Thus, the 5′ phosphorylated complementary strand of the amplification product will be digested by the exonuclease, generating a single-stranded amplification product that is suitable for hybridization. In certain embodiments, the exonuclease digests all or a part of one strand of an amplification product.

According to certain embodiments, the probes of the present invention comprise a target-specific portion, an addressable support-specific portion, and a primer-specific portion (see, e.g., probe 2 of FIG. 2). The probe's target-specific portion is designed to specifically hybridize with a complementary region of the target nucleic acid sequence. The addressable support-specific portion may, but need not be located between the primer-specific portion and the target-specific portion (see, for example, probe 23 in FIG. 3). In certain embodiments, the probe's addressable support-specific portion is not complementary with the target or primer sequences. The addressable support-specific portion, or its complement, is designed to specifically hybridize directly, indirectly, or both with an addressable support or to have a mobility such that it is located at a particular mobility address during or after appropriate separation procedures, such as an MDAT.

In certain embodiments, the methods of the invention comprise universal primers, universal primer sets, or both. In certain embodiments, 5′ primer-specific portions of at least two different ligation products comprise a sequence that is the same as at least a portion of one first primer in the reaction mixture (see, e.g., primer PA in FIG. 9(a)). Similarly, at least two different ligation products in a reaction mixture comprise a 3′ primer-specific portion that is complementary to at least a portion of one second primer (see, e.g., primer PZ in FIG. 9(a)). In certain embodiments, the 5′ primer-specific portions of most ligation products in a reaction mixture comprise a sequence that is the same as the at least one first primer, and the 3′ primer-specific portions of most of the ligation products in a reaction mixture comprise a sequence that is complementary to at least one second primer (see, e.g., primers PA and PZ in FIG. 9(b)). In certain embodiments, the 5′ primer-specific portions of all ligation products in a reaction mixture comprise a sequence that is the same as the at least one first primer, and the 3′ primer-specific portions of all of the ligation products in a reaction mixture comprise a sequence that is complementary to at least one second primer (see, e.g., primers PA and PZ in FIG. 9(c)). In certain embodiments, a reaction mixture comprises more than one universal primer, more than one universal primer set, or both.

Such ligation products can be used in, for example, but are not limited to, a multiplex reaction wherein multiple target nucleic acid sequences are quantitated. According to certain embodiments, at least one universal primer, at least one universal primer set, or both, are used in a multiplex reaction to obtain quantitative results useful in gene expression profiling.

According to certain embodiments, a multiplex reaction may include, for example, but is not limited to, six ligation products, each comprising a unique addressable support-specific portion corresponding to different target sequences or alleles or a combination of both (see, e.g., FIG. 9). In FIG. 9(a), the 5′ primer-specific portions of two ligation products (A-Z) comprise a sequence that is the same as at least a portion of one first primer (PA) in the reaction mixture. The 3′ primer-specific portions of the same two ligation products comprise a sequence that is complementary to at least a portion of one second primer in the reaction mixture. Thus, to exponentially amplify these six ligation products, one uses five primer sets (PA-PZ, PC-PX, PD-PW, PE-PV, and PF-PU).

FIG. 9(b) shows the same six ligation products, except that the 5′ primer-specific portions of most of the ligation products comprise a sequence that is the same as at least a portion of one first primer in the reaction mixture. The 3′ primer-specific portions of most of the ligation products comprise a sequence that is complementary to at least a portion of one second primer in the reaction mixture. To exponentially amplify these six ligation products, three primer sets are used (PA-PZ, PE-PV, and PF-PU).

FIG. 9(c) shows the same six ligation products, except that the 5′ primer-specific portions of all of the ligation products comprise a sequence that is the same as at least a portion of one first primer in the reaction mixture. The 3′ primer-specific portions of all of the ligation products comprise a sequence that is complementary to at least a portion of one second primer in the reaction mixture. To exponentially amplify these six ligation products, only one primer set is used (PA-PZ).

Thus, the same primer set will be used for at least two ligation products in the reaction mixture (see, e.g., primers PA and PZ of FIG. 9(a)). In certain embodiments, most ligation products in the reaction mixture will use the same primer set (see, e.g., primers PA and PZ of FIG. 9(b)). In certain embodiments, all of the ligation products in the reaction mixture will use the same primer set (see, e.g., primers PA and PZ of FIG. 9(c)).

According to the present invention, as few as one universal primer or one universal primer set can be used to amplify an infinite number of ligation or amplification products, since the probes may be designed to share primer-specific portions but comprise different addressable support-specific portions.

The methods of the instant invention according to certain embodiments may comprise universal primers or universal primer sets that decrease the number of different primers that are added to the reaction mixture, reducing the cost and time required. For example, without limitation, in a 100 target sequence multiplex reaction, typically 100 different primer sets are required using certain conventional methods. According to certain embodiments of the invention, anywhere from 100 primer sets to as few as one primer set may be employed in the same 100 target multiplex. For example, in certain embodiments, all of the ligation or amplification products to be amplified by a universal primer or universal primer set comprise the same 5′ primer-specific portion and the same 3′ primer-specific portion. The skilled artisan will appreciate that more than one universal primer set may be employed in a multiplex reaction, each specific to a different subset of ligation or amplification products in the reaction. In certain embodiments, the amplification reaction mixture may comprise at least one universal primer or universal primer set and at least one primer or primer set that hybridizes to only one species of probe, ligation product, or amplification product.

Because only one or a limited number of primers or primer sets are required for amplification according to certain embodiments, the methods are more cost-efficient and less time-consuming than conventional methods of quantitating target nucleic acid sequences in a sample. Using a limited number of primers may also reduce variation in amplification efficiency and cross-reactivity of the primers in certain embodiments. Additionally, quantitative results may be obtained from multiplex reactions for those ligation products or amplification products that are amplified by a universal primer or universal primer set, respectively.

The skilled artisan will appreciate, however, that in certain embodiments, including, but not limited to, detecting multiple alleles, the ligation reaction mixture may comprise more than one first probe or more than one second probe for each potential allele in a multiallelic target locus. Those methods preferably employ more than one first primer or more than one second primer in a reaction mixture. For example, one first primer for all first alleles to be detected, a different first primer for all second alleles to be detected, another first primer for all third alleles to be detected, and so forth.

The significance of the decrease in the number of primers, and therefore the cost and number of manipulations required, becomes readily apparent when performing genetic screening of an individual for a large number of multiallelic loci. In certain embodiments, one may use, for example, without limitation, a simple screening assay to detect the presence of three biallelic loci (e.g., L1, L2, and L3) in an individual using three probe sets. See, e.g., Table 1 below.

TABLE 1

| | | | | Addressable |
|---|---|---|---|---|
| | | | | Support-Specific |
| Locus | Allele | Probe Set | Primer Set | Portion |
| L1 | 1 | A1, Z1 | PA, PZ | 1 |
| | 2 | B1, Z1 | PB, PZ | 2 |
| L2 | 1 | A2, Z2 | PA, PZ | 3 |
| | 2 | B2, Z2 | PB, PZ | 4 |
| L3 | 1 | A3, Z3 | PA, PZ | 5 |
| | 2 | B3, Z3 | PB, PZ | 6 |

For illustration purposes, each of the three probe sets comprise two first probes, for example, A and B, and one second probe, Z. Both first probes, A and B, comprise the same upstream target-specific sequence, but differ at the pivotal complement. The skilled artisan, however, will understand that the probes can be designed with the pivotal complement at any location in either the first probe or the second probe. Additionally, probes comprising multiple pivotal complements are within the scope of the invention.

To distinguish between the two possible alleles in each biallelic locus, probes A and B comprise different 5′ primer-specific sequences. Therefore, two different first primers, PA and PB, hybridize with the complement of the primer-specific portions of probe A and probe B, respectively. A third primer, PZ, hybridizes with the primer-specific portion of probe Z. If the different first primers comprise different reporter groups, the reporter groups can be used to distinguish between the allele-specific ligation products. Thus, in these embodiments three probes A1, B1, and Z1, are used to form the two possible L1 ligation products, wherein A1Z1 is the ligation product of the first L1 allele and B1Z1 is the ligation product of the second L1 allele. Likewise, probes A2, B2, and Z2, are used to form the two possible L2 ligation products. Probe A2 comprises the same primer-specific portion as probe A1, the primer-specific portion of probe B2 is the same as probe B1, and so forth. Thus, as few as three primers, PA, PB, and PZ, could be used in these embodiments. According to these embodiments, the detection of only one label at the capture oligonucleotide or at a particular mobility location would indicate that the sample was obtained from a homozygous individual. Both labels would be detected at the capture oligonucleotide or mobility location if the sample was obtained from a heterozygous individual.

In these embodiments, the number of probes needed to detect any number of target sequences, therefore, is the product of the number of targets to be detected times the number of alleles to be detected per target plus one (i.e., (number of target sequences×[number of alleles+1]). Thus, to detect 3 biallelic sequences, for example, nine probes are needed (3×[2+1]), or as shown in Table 1, (A1, B1, Z1, A2, B2, Z2, A3, B3, and Z3). To detect 4 triallelic sequences 16 probes are needed (4×[3+1]), and so forth.

In these embodiments, to amplify the ligation product of target sequence L1, three primers are needed to address a biallelic locus, PA, complementary to the 5′ primer-specific portion of A1; PB, complementary to the 5′ primer-specific portion of B1; and PZ, complementary to the 3′ primer-specific portion of Z1, respectively. To amplify the ligation product of target sequence L2, using certain conventional methods, three additional primers are required, e.g., PA2, PB2, and PZ2; likewise to amplify target sequence L3, requires yet three more primers, PA3, PB3, and PZ3. Thus, to amplify the ligation products for three biallelic loci potentially present in an individual using certain conventional methodology, would require 9 (3n, where n=3) primers.

In contrast, the methods of the present invention can effectively reduce this number to as few as three amplification primers in this example. Using the present invention, one can use at least two different A probes that comprise the same 5′ primer-specific sequence. More preferably, most of the different A probes comprise the same 5′ primer-specific sequence. Most preferably, all of the different A probes comprise the same 5′ primer-specific sequence. Similarly, at least two, more preferably most, and most preferably all of the different B probes comprise the same 5′ primer-specific sequence. Finally, at least two, more preferably most, and most preferably all of the different Z probes comprise the same 3′ primer-specific sequence. Thus, as few as one A primer, one B primer, and one Z primer can be used to amplify all of ligation products (PA, PB and PZ in Table 1).

In other embodiments, one can use different addressable support-specific portions to distinguish between the allele-specific ligation products. Thus, for a biallelic locus, for example, but without limitation, the same first labeled primer can be used to hybridize with the complement of either probe A or probe B. A second primer, PZ, hybridizes with the primer-specific portion of probe Z. Thus, as few as two primers could be used in these embodiments. According to these embodiments, the detection of only a single labeled amplification product hybridized to its respective capture oligonucleotide or at a mobility location would indicate that the sample was obtained from a homozygous individual. If the sample was obtained from a heterozygous individual, both amplification products would hybridize with their respective capture oligonucleotides or be detected at appropriate mobility locations.

According to the present invention, as few as two or three “universal” primers, can be used to amplify an infinite number of ligation or amplification products, since the probes may be designed to share primer-specific portions but comprise different addressable support-specific portions.

Rather than the nine primers required to detect all potential alleles in three biallelic loci, using certain conventional methodology (e.g., PA1, PB1, PZ1, PA2, PB2, PZ2, PA3, PB3, and PZ3), the methods of the present invention can use as few as three primers (PA, PB, and PZ, as shown in Table 1). A sample containing 100 possible biallelic loci would require 200 primers in certain conventional detection methods, yet only 3 universal primers can be used in the instant methods. This dramatic decrease in the number of required amplification primers is possible since at least one probe in each probe set has the addressable support-specific portion located between the primer-specific portion and the target-specific portion.

In certain embodiments, different alleles in a multiallelic locus are differentiated using primers with different reporter groups. For example, but without limitation, if the first allele is present in the sample, the ligation product will comprise primer-specific portion A. If the second allele is present in the sample, the ligation product will comprise primer-specific portion B. In certain embodiments, primer PA, complementary to portion A, comprises a green reporter group, while primer PB, complementary to portion B, comprises a red reporter group. The two alleles are differentiated by detecting either a green or a red reporter group hybridized via the addressable support-specific portion to the support at a spatially addressable position or at a mobility location. Both the green and the red reporter groups will be detected if the individual is heterozygous for the biallelic target locus.

In other embodiments, different alleles in a multiallelic locus are differentiated using probes with different addressable-support-specific portions. For example, but without limitation, if the first allele is present in the sample, the ligation product will comprise addressable support-specific portion A. If the second allele is present in the sample, the ligation product will comprise addressable support-specific portion B. At least one primer for each ligation product comprises a red reporter group. The two alleles are differentiated by detecting a red reporter group hybridized with the support at one of two spatially addressable positions or mobility locations. The person of ordinary skill will appreciate that three or more alleles at a multiallelic locus can also be differentiated using these methods.

In certain embodiments, different reporter groups and different addressable support-specific portions are combined to distinguish different target nucleic acid sequences. In certain embodiments, the at least one first probes and the at least one second probes in a probe set comprise different reporter groups.

In certain embodiments, different amplification products are detected by mobility discrimination using separation techniques such as electrophoresis, mass spectroscopy, or chromatography rather than hybridization to capture oligonucleotides on a support. In certain embodiments, the addressable support-specific portions may have uniquely identifiable lengths or molecular weights. Alternatively, an addressable support-specific portion may be complementary to a particular mobility-modifier comprising a tag complement for selectively binding to the addressable support-specific portion of the amplification product, and a tail for effecting a particular mobility in a mobility-dependent analysis technique, e.g., electrophoresis, e.g., U.S. patent application Ser. No. 09/522,640, filed Mar. 15, 1999. Thus, the amplification products can be separated by molecular weight or length to distinguish the individual amplified sequences. The detection of an amplification product in a particular molecular weight or length bin indicates the presence of the corresponding target nucleic acid sequence in the starting material. Descriptions of mobility discrimination techniques may be found, among other places, in U.S. Pat. Nos. 5,470,705, 5,514,543, 5,580,732, 5,624,800, and 5,807,682.

In an exemplary protocol, air-dried amplification pellets, comprising amplification products of uniquely identifiable molecular weight, are resuspended in buffer or deionized formamide. The resuspended samples and a molecular weight marker (e.g., GS 500 size standard, Applied Biosystems, Foster City, Calif.) are loaded onto an electrophoresis platform (e.g., ABI Prism™ Genetic Analyzer, Applied Biosystems) and electrophoresed in POP-4 polymer (Applied Biosystems) at 15 kV using a 50 μl capillary. The bands are detected, quantitated, and their position relative to the marker is determined. The bands are identified based on their relative electrophoretic mobility, indicating the presence of their respective target sequence in the sample. The bands may be quantitated, for example, based on the relative intensity of the associated reporter group.

Alternatively, each addressable support-specific portion contains a sequence that is complementary to a mobility-modifier comprising a tag complement that is complementary to the addressable support-specific portion of the amplification product, and a tail, for effecting a particular mobility in a mobility-dependent analysis technique (MDAT), e.g., electrophoresis, such that when the tag complement and the addressable support-specific portion are contacted a stable complex is formed, see, e.g., U.S. patent application Ser. No. 09/522,640 filed Mar. 15, 1999. As used herein, “mobility-dependent analysis technique” or MDAT means an analytical technique based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques, and the like.

According to certain embodiments of the invention, certain addressable support-specific portions and tag-complements should form a complex that (1) is stable under conditions typically used in nucleic acid analysis methods, e.g., aqueous, buffered solutions at room temperature; (2) is stable under mild nucleic-acid denaturing conditions; and (3) does not adversely effect the sequence specific binding of a target-specific portion of a probe with a target nucleic acid sequence. In addition, in certain embodiments, addressable support-specific portions and tag complements of the invention should accommodate sets of distinguishable addressable support-specific portions and tag complements such that a plurality of different amplification products and associated mobility modifiers may be present in the same reaction volume without causing cross-interactions among the addressable support-specific portions, tag complements, target nucleic acid sequence and target-specific portions of the probes. Certain methods for selecting sets of tag sequences that minimally cross hybridize are described elsewhere (e.g., Brenner and Albrecht, PCT Patent Application No. WO 96/4101 1).

In certain embodiments, the addressable support-specific portions and tag complement each comprise polynucleotides. In certain embodiments, the polynucleotide tag complements are rendered non-extendible by a polymerase, e.g., by including sugar modifications such as a 3′-phosphate, a 3′-acetyl, a 2′-3′-dideoxy, a 3′-amino, and a 2′-3′ dihydro.

In certain embodiments, an addressable support-specific portion and tag complement pair comprises an addressable support-specific portion that is a conventional synthetic polynucleotide, and a tag complement that is PNA. Where the PNA tag complement has been designed to form a triplex structure with a tag, the tag complement may include a “hinge” region in order to facilitate triplex binding between the tag and tag complement. In certain embodiments, addressable support-specific portions and tag complement sequences comprise repeating sequences. Such repeating sequences in the addressable support-specific portions and tag complement are used in certain embodiments for their (1) high binding affinity, (2) high binding specificity, and (3) high solubility. An exemplary repeating sequence for use as a duplex-forming addressable support-specific portions or tag complement is (CAG)ₙ, where the three base sequence is repeated from about 1 to 10 times (see, e.g., Boffa, et al., PNAS (USA), 92:1901-05 (1995); Wittung, et al., Biochemistry, 36:7973-79 (1997)). An exemplary repeating sequence for use as a triplex-forming addressable support-specific portions or tag complement is (TCC)ₙ.

PNA and PNA/DNA chimera molecules can be synthesized using well known methods on commercially available, automated synthesizers, with commercially available reagents (see, e.g., Dueholm, et al., J. Org. Chem., 59:5767-73 (1994); Vinayak, et al., Nucleosides & Nucleotides, 16:1653-56 (1997)).

In certain embodiments, the addressable support-specific portion may comprise all, part, or none of the target-specific portion of the probe. In certain embodiments, the addressable support-specific portion may consist of some or all of the target-specific portion of the probe. In certain embodiments, the addressable support-specific portions do not comprise any portion of the target-specific portion of the probe.

In certain embodiments, the mobility-modifier of the present invention comprises a tag complement portion for binding to the addressable support-specific portion of the amplification product, and a tail for effecting a particular mobility in a mobility-dependent analysis technique.

The tail portion of a mobility modifier may be any entity capable of effecting a particular mobility of a amplification product/mobility-modifier complex in a mobility-dependent analysis technique. In certain embodiments, the tail portion of the mobility modifier of the invention should (1) have a low polydispersity in order to effect a well-defined and easily resolved mobility, e.g., Mw/Mn less than 1.05; (2) be soluble in an aqueous medium; (3) not adversely affect probe-target hybridization or addressable support-specific portion/tag complement binding; and (4) be available in sets such that members of different sets impart distinguishable mobilities to their associated complexes.

In certain embodiments, the tail portion of the mobility modifier comprises a polymer. Specifically, the polymer forming the tail may be homopolymer, random copolymer, or block copolymer. Furthermore, the polymer may have a linear, comb, branched, or dendritic architecture. In addition, although the invention is described herein with respect to a single polymer chain attached to an associated mobility modifier at a single point, the invention also contemplates mobility modifiers comprising more than one polymer chain element, where the elements collectively form a tail portion.

Exemplary polymers for use in the present invention include, but are not limited to, hydrophilic, or at least sufficiently hydrophilic when bound to a tag complement to ensure that the tag complement is readily soluble in aqueous medium. Where the mobility-dependent analysis technique is electrophoresis in certain embodiments, the polymers are uncharged or have a charge/subunit density that is substantially less than that of the amplification product.

In certain embodiments, the polymer is polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of N 12 mer PEO units, and a chain composed of N tetrapeptide units, where N is an adjustable integer (e.g., Grossman et al., U.S. Pat. No. 5,777,096).

In certain embodiments, the synthesis of polymers useful as tail portions of a mobility modifier of the present invention may depend on the nature of the polymer. Methods for preparing suitable polymers generally follow well known polymer subunit synthesis methods. Methods of forming selected-length PEO chains are discussed below. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J. Peptide Protein Res., 35:161-214 (1990)).

In certain methods for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., Levenson et al., U.S. Pat. No. 4,914,210).

Another exemplary polymer for use as a tail portion is PNA. Certain advantages, properties and synthesis of PNA have been described above. In particular, when used in the context of a MDAT comprising an electrophoretic separation in free solution, PNA has the advantageous property of being essentially uncharged.

Coupling of the polymer tails to a polynucleotide tag complement can be carried out by an extension of conventional phosphoramidite polynucleotide synthesis methods, or by other standard coupling methods, e.g., a bis-urethane tolyl-linked polymer chain may be linked to a polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods.

As noted above, the tail portion of the mobility modifier imparts a mobility to a amplification product/mobility modifier complex that is distinctive for each different probe/mobility modifier complex. The contribution of the tail to the mobility of the complex in certain embodiments, will generally depend on the size of the tail. However, addition of charged groups to the tail, e.g., charged linking groups in the PEO chain, or charged amino acids in a polypeptide chain, can also be used to achieve selected mobility characteristics in the probe/mobility modifier complex. It will also be appreciated that the mobility of a complex may be influenced by the properties of the amplification product itself, e.g., in electrophoresis in a sieving medium, a larger probe in certain embodiments, will reduce the electrophoretic mobility of the probe/mobility modifier complex.

The tag complement portion of a mobility modifier according to the present invention may be any entity capable of binding to, and forming a complex with, an addressable support-specific portion of an amplification product. Furthermore, the tag-complement portion of the mobility modifier may be attached to the tail portion using conventional means.

When a tag complement is a polynucleotide, e.g., PNA, the tag complement may comprise all, part, or none of the tail portion of the mobility modifier. In certain embodiments of the invention, the tag complement may consist of some or all of the tail portion of the mobility modifier. In other embodiments of the invention, the tag complement does not comprise any portion of the tail portion of the mobility modifier. For example, because PNA is uncharged, particularly when using free solution electrophoresis as the mobility-dependent analysis technique, the same PNA oligomer may act as both a tag complement and a tail portion of a mobility modifier.

In certain embodiments, the tag complement includes a hybridization enhancer, where, as used herein, the term “hybridization enhancer” means moieties that serve to enhance, stabilize, or otherwise positively influence hybridization between two polynucleotides, e.g. intercalators (e.g., U.S. Pat. No. 4,835,263), minor-groove binders (e.g., U.S. Pat. No. 5,801,155), and cross-linking functional groups. The hybridization enhancer may be attached to any portion of a mobility modifier, so long as it is attached to the mobility modifier is such a way as to allow interaction with the addressable support-specific portion/tag complement duplex. However, in certain embodiments, the hybridization enhancer is covalently attached to a mobility modifier of the binary composition. In certain embodiments, a hybridization enhancer for use in the present invention is minor-groove binder, e.g., netropsin, distamycin, and the like.

In certain embodiments, a plurality of amplification product/mobility modifier complexes are resolved via a MDAT.

In one embodiment of the invention, amplification product/mobility modifier complexes are resolved (separated) by liquid chromatography and quantitated. Exemplary stationary phase media for use in the method include reversed-phase media (e.g., C-18 or C-8 solid phases), ion-exchange media (particularly anion-exchange media), and hydrophobic interaction media. In a related embodiment, the amplification product/mobility modifier complexes can be separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating polynucleotides, an ion-pairing agent (e.g., a tetra-alkylammonium) is typically included in the solvent to mask the charge of phosphate.

The mobility of an amplification product/mobility modifier complex can be varied by using mobility modifiers comprising polymer chains that alter the affinity of the probe for the solid, or stationary, phase. Thus, with reversed-phase chromatography, an increased affinity of the amplification product/mobility modifier complexes for the stationary phase can be attained by addition of a moderately hydrophobic tail (e.g., PEO-containing polymers, short polypeptides, and the like) to the mobility modifier. Longer tails impart greater affinity for the solid phase, and thus one may use higher non-polar solvent concentration for the probe to be eluted (and a longer elution time).

According to certain embodiments of the present invention, the amplification product/mobility modifier complexes are resolved by electrophoresis in a sieving or non-sieving matrix and quantitated. In certain embodiments, the electrophoretic separation is carried out in a capillary tube by capillary electrophoresis (see, e.g., Capillary Electrophoresis: Theory and Practice, Grossman and Colburn eds., Academic Press (1992)). Sieving matrices that may be used include covalently crosslinked matrices, such as polyacrylamide covalently crosslinked with bis-acrylamide; gel matrices formed with linear polymers (e.g., Madabhushi et al. U.S. Pat. No. 5,552,028); and gel-free sieving media (e.g., Grossman et al., U.S. Pat. No. 5,624,800; Hubert and Slater, Electrophoresis, 16: 2137-2142 (1995); Mayer et al., Analytical Chemistry, 66(10): 1777-1780 (1994)). The electrophoresis medium may contain a nucleic acid denaturant, such as 7M formamide, for maintaining polynucleotides in single-stranded form. Suitable capillary electrophoresis instrumentation are commercially available, e.g., the ABI PRISM™ Genetic Analyzer (Applied Biosystems).

The skilled artisan will appreciate that the amplification products can also be separated based on molecular weight, length, or mobility by, for example, but without limitation, gel filtration, mass spectroscopy, or HPLC, and detected and quantitated using appropriate methods.

In certain embodiments, for each target nucleic acid sequence to be detected and quantitated at least one probe set, comprising at least one first probe and at least one second probe, is combined with the sample to form a ligation reaction mixture (see, e.g., FIG. 2A). In certain embodiments, the ligation reaction mixture further comprises a ligation agent. In certain embodiments, either the at least one first probe or the at least one second probe comprises an addressable support-specific portion, located between the primer-specific portion and the target-specific portion. See, for example probe 23 in FIG. 3, which includes an addressable support-specific portion 4 located between the primer-specific portion 5 and the target-specific portion 15b. In certain embodiments, the addressable support-specific portion may be identifiable by molecular weight, length, or mobility, or may be complementary to a particular mobility modifier. For example, without limitation, the addressable support-specific portion that corresponds to one target nucleic acid sequence will be 2 nucleotides in length, the addressable support-specific portion that corresponds to a second target nucleic acid sequence will be 4 nucleotides in length, the addressable support-specific portion that corresponds to a third target nucleic acid sequence will be 6 nucleotides in length, and so forth. In certain embodiments, the addressable support-specific portion will be less than 101 nucleotides (i.e., 0 to 100 nucleotides) long, less than 41 nucleotides (i.e., 0 to 40 nucleotides) long, or 2 to 36 nucleotides long. In certain embodiments, the addressable support-specific portion that correspond to a particular target nucleic acid sequence will differ in length from the addressable support-specific portions that correspond to different target sequences by at least two nucleotides.

Following at least one amplification cycle, the amplification products are separated based on their molecular weight or length or mobility by, for example, without limitation, gel electrophoresis, HPLC, MALDI-TOF, gel filtration, or mass spectroscopy. The detection and quantitation of a labeled sequence at a particular mobility address indicates that the sample or starting material contains the corresponding target nucleic acid sequence at the determined concentration.

In certain embodiments, the first and second probes in each probe set are designed to be complementary to the sequences immediately flanking the pivotal nucleotide of the target sequence. Either the at least one first probe or the at least one second probe of a probe set, but not both, will comprise the pivotal complement. When the target sequence is present in the sample, the first and second probes will hybridize, under appropriate conditions, to adjacent regions on the target. When the pivotal complement is base-paired in the presence of an appropriate ligation agent, two adjacently hybridized probes may be ligated together to form a ligation product. Alternatively, under appropriate conditions, autoligation may occur. The skilled artisan will appreciate that the pivotal nucleotide(s) may be located anywhere in the target sequence and that likewise, the pivotal complement may be located anywhere within the target-specific portion of the probe(s).

The ligation reaction mixture (in the appropriate salts, buffers, and nucleotide triphosphates) is then combined with at least one primer set and a polymerase to form a first amplification reaction mixture. In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement. When the ligation product exists as a double-stranded molecule, subsequent amplification cycles may exponentially amplify this molecule.

The primer set comprises at least one reporter group so that the amplification products resulting from incorporation of these primers will include a reporter group specific for the particular pivotal nucleotide that is included in the original target sequence.

Following at least one amplification cycle, the amplification products are separated based on their molecular weight or length or mobility by, for example, without limitation, gel electrophoresis, HPLC, MALDI-TOF, gel filtration, or mass spectroscopy. The detection of a labeled sequence at a particular mobility address indicates that the sample contains the related target sequence.

According to certain embodiments, the present invention may be used to identify and quantify splice variants in a target nucleic acid sequence. For example, genes, the DNA that encodes for a protein or proteins, may contain a series of coding regions, referred to as exons, interspersed by non-coding regions referred to as introns. In a splicing process, introns are removed and exons are juxtaposed so that the final RNA molecule, typically a messenger RNA (mRNA), comprises a continuous coding sequence. While some genes encode a single protein or polypeptide, other genes can code for a multitude of proteins or polypeptides due to alternate splicing.

For example, a gene may comprise five exons each separated from the other exons by at least one intron, see FIG. 10. The hypothetical gene that encodes the primary transcript, shown at the top of FIG. 10, codes for three different proteins, each encoded by one of the three mature mRNAs, shown at the bottom of FIG. 10. Due to alternate splicing, exon 1 may be juxtaposed with (a) exon 2a-exon 3, (b) exon 2b-exon 3, or (c) exon 2c-exon 3, the three splicing options depicted in FIG. 10, which result in the three different versions of mature mRNA.

The rat muscle protein, troponin T is but one example of alternate splicing. The gene encoding troponin T comprises five exons (W, X, α, β, and Z), each encoding a domain of the final protein. The five exons are separated by introns. Two different proteins, an α-form and a β-form are produced by alternate splicing of the troponin T gene. The α-form is translated from a mRNA that contains exons W, X, α, and Z. The β-form is translated from a mRNA that contains exons W, X, β, and Z.

In certain embodiments, a method is provided for identifying and quantifying splice variants in at least one target nucleic acid sequence in a sample comprising combining at least one target nucleic acid sequence with a probe set for each target nucleic acid sequence to form a ligation reaction mixture. In certain embodiments, the probe set comprises (a) at least one first probe, comprising a target specific portion and a 5′ primer-specific portion; and (b) a plurality of second probes, each second probe comprising a 3′ primer-specific portion and one of a plurality of splice-specific portions. In certain embodiments, at least one probe in each probe set further comprises at least one addressable support-specific portion located between the primer-specific portion and the target-specific portion, or between the primer-specific portion and the splice-specific portion. The probes in each probe set are suitable for ligation together when hybridized adjacent to one another on a target sequence. In certain embodiments, the ligation reaction mixture further comprises a ligation agent.

In certain embodiments, the ligation reaction mixture is subjected to at least one cycle of ligation, wherein adjacently hybridized probes are ligated together to form a ligation product comprising the 5′ primer-specific portion, the target-specific portion, the splice-specific portion, the at least one addressable support-specific portion, and the 3′ primer-specific portion. In certain embodiments, this ligation reaction mixture is combined with at least one primer set comprising at least one first primer comprising the sequence of the 5′ primer-specific portion of the ligation product and at least one second primer comprising a sequence complementary to the 3′ primer-specific portion of the ligation product, wherein at least one primer of the primer set further comprises a reporter group and a polymerase to form a first amplification reaction mixture.

In certain embodiments, a first amplification product, comprising at least one reporter group, is generated by subjecting the first amplification reaction mixture to at least one amplification cycle. The first amplification product or a portion of the first amplification product comprising at least one reporter group is analyzed using at least a portion of the at least one addressable support-specific portion. In certain embodiments, the identity of the splice variant is determined by detecting the at least one reporter group that is hybridized to a specific address on an addressable support or located in a specific mobility address. The quantity of the splice variant in the at least one target nucleic acid sequence is determined.

In certain embodiments, a method is provided for identifying and quantifying splice variants in at least one target nucleic acid sequence in a sample comprising combining at least one target nucleic acid sequence with a probe set for each target nucleic acid sequence to form a ligation reaction mixture. In certain embodiments, the probe set comprises (a) at least one first probe, comprising a target specific portion and (b) a plurality of second probes, each second probe comprising a 3′ primer-specific portion and one of a plurality of splice-specific portions. At least one probe in each probe set further comprises at least one addressable support-specific portion. The probes in each probe set are suitable for ligation together when hybridized adjacent to one another on a target sequence. In certain embodiments, the ligation reaction mixture further comprises a ligation agent.

In certain embodiments, the ligation reaction mixture is subjected to at least one cycle of ligation, wherein adjacently hybridized probes are ligated together to form a ligation product comprising the target-specific portion, the splice-specific portion, the at least one addressable support-specific portion, and the 3′ primer-specific portion. In certain embodiments, this ligation reaction mixture is combined with at least one primer set comprising at least one primer comprising a sequence complementary to the 3′ primer-specific portion of the ligation product, wherein at least one primer of the primer set further comprises a reporter group and a polymerase to form an extension reaction mixture.

In certain embodiments, a first amplification product, comprising at least one reporter group, is generated by subjecting the first amplification composition to at least one cycle of primer extension. The first amplification product or a portion of the first amplification product comprising at least one reporter group is analyzed using at least a portion of the at least one addressable support-specific portion. In certain embodiments, the identity of the splice variant is determined by detecting the at least one reporter group that is hybridized to a specific address on an addressable support or located in a specific mobility address. The quantity of the splice variant in the at least one target nucleic acid sequence is determined.

In certain embodiments, a method is provided for identifying and quantifying splice variants in at least one target nucleic acid sequence in a sample comprising combining at least one target nucleic acid sequence with a probe set for each target nucleic acid sequence to form a ligation reaction mixture. In certain embodiments, the probe set comprises (a) at least one first probe, comprising a target specific portion and a 5′ primer-specific portion; and (b) a plurality of second probes, each second probe comprising a 3′ primer-specific portion and one of a plurality of splice-specific portions. In certain embodiments, at least one probe in each probe set further comprises at least one addressable support-specific portion located between the primer-specific portion and the target-specific portion, or between the primer-specific portion and the splice-specific portion. The probes in each probe set are suitable for ligation together when hybridized adjacent to one another on a target sequence. In certain embodiments, the ligation reaction mixture further comprises a ligation agent.

In certain embodiments, the ligation reaction mixture is subjected to at least one cycle of ligation, wherein adjacently hybridized probes are ligated together to form a ligation product comprising the 5′ primer-specific portion, the target-specific portion, the splice-specific portion, the at least one addressable support-specific portion, and the 3′ primer-specific portion. In certain embodiments, this ligation reaction mixture is combined with at least one primer set comprising at least one first primer comprising the sequence of the 5′ primer-specific portion of the ligation product and at least one second primer comprising a sequence complementary to the 3′ primer-specific portion of the ligation product, and a polymerase to form a first amplification reaction mixture.

In certain embodiments, a first amplification product is generated by subjecting the first amplification composition to at least one amplification cycle. In certain embodiments, a second amplification reaction mixture is formed by combining the first amplification product with either at least one first primer, or at least one second primer for each primer set, but not both first and second primers, wherein the at least one first primer or the at least one second primer for each primer set further comprises a reporter group. In certain embodiments, a second amplification product comprising the at least one reporter group is generated by subjecting the second amplification reaction mixture to at least one cycle of amplification.

The second amplification product or a portion of the second amplification product comprising at least one reporter group is analyzed using at least a portion of the at least one addressable support-specific portion. In certain embodiments, the identity of the splice variant is determined by detecting the at least one reporter group that is hybridized to a specific address on an addressable support or located in a specific mobility address. The quantity of the splice variant in the at least one target nucleic acid sequence is determined.

In certain embodiments, the at least one target nucleic acid sequence comprises at least one complementary DNA (cDNA) generated from an RNA. In certain embodiments, the at least one cDNA is generated from at least one messenger RNA (mRNA). In certain embodiments, the at least one target nucleic acid sequence comprises at least one RNA target sequence present in the sample.

In certain embodiments, the ligation reaction compostion further comprises a ligation agent, such as, but not limited to T4 DNA ligase, or thermostable ligases such as, but not limited to, Tth ligase, Taq ligase, Tsc ligase, or Pfu ligase. In certain embodiments, the polymerase of the amplification reaction mixture is a DNA-dependent DNA polymerase. In certain embodiments the DNA-dependent DNA polymerase is a thermostable polymerase, for example, but not limited to, Taq polymerase, Pfx polymerase, Pfu polymerase, Vent® polymerase, Deep Vent™ polymerase, Pwo polymerase, or Tth polymerase.

In certain embodiments, the at least one reporter group comprises a fluorescent moiety. In certain embodiments, the molar concentration of the at least one first primer is different from the molar concentration of the at least one second primer in the at least one primer set. In certain embodiments, in at least one primer set, the melting temperature (Tm₅₀) of the at least one first primer differs from the melting temperature of the at least one second primer by at least about 4° C., by at least about 8° C., by at least about 10° C., or by at least about 12° C.

In various embodiments for identifying and quantifying splice variants, one can use any of the various embodiments employing addressable support-specific portions disclosed in this application. In various embodiments for identifying splice variants, one can use any of the various embodiments employing primer specific portions disclosed in this application. Also, if one desires to identify and quantify but one splice variant, they can use only one second probe comprising a splice-specific portion (specific to that one splice variant).

Certain nonlimiting embodiments for identifying splice variants are illustrated by FIG. 11. Such embodiments permit one to identify and quantify two different splice variants. One splice variant includes exon 1, exon 2, and exon 4. The other splice variant includes exon 1, exon 3, and exon 4. In such embodiments, one can use the same addressable support-specific portion for both variants and the variants may be distinguished based on a color signal. The target specific portion corresponds to at least a portion of exon 1. The splice-specific portions correspond to at least a portion of the specific exon (exon 2 or exon 3). The skilled artisan will understand that PSPa, PSPb, or PSPc may be the 5′ primer-specific portion or the ′3 primer-specific portion depending on the orientation of the target sequence.

Exemplary Kits

In certain embodiments, the invention also provides kits designed to expedite performing the subject methods. Kits serve to expedite the performance of the methods of interest by assembling two or more components used in carrying out the methods. Kits may contain components in pre-measured unit amounts to minimize the need for measurements by end-users. Kits may include instructions for performing one or more methods of the invention. In certain embodiments, the kit components are optimized to operate in conjunction with one another.

According to certain embodiments, kits for quantitating at least one target nucleic acid sequence in a sample are provided. In certain embodiments, the kits comprise at least one probe set comprising (a) at least one first probe, comprising a first target-specific portion and a 5′ primer-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one probe in each probe set further comprises at least one addressable support-specific portion located between the primer-specific portion and the target-specific portion of the at least one probe in each probe set.

According to certain embodiments, kits for quantitating at least one target nucleic acid sequence in a sample are provided. In certain embodiments, the kits comprise at least one probe set comprising (a) at least one first probe, comprising a first target-specific portion, and (b) at least one second probe, comprising a second target-specific portion and a 3′ primer-specific portion. In certain embodiments, the probes in each set are suitable for ligation together when hybridized adjacent to one another on the at least one target nucleic acid sequence. In certain embodiments, at least one second probe in each probe set further comprises at least one addressable support-specific portion located between the primer-specific portion and the target-specific portion of the at least one second probe in each probe set.

The kits of the invention may comprise components such as at least one polymerase, at least one transcriptase, at least one ligation agent, oligonucleotide triphosphates, nucleotide analogs, reaction buffers, salts, ions, stabilizers, or combinations of these components. Certain kits of the invention comprise reagents for purifying the ligation products, including, without limitation, dialysis membranes, chromatographic compounds, supports, oligonucleotides, or combinations of these reagents.

The following examples are intended for illustration purposes only, and should not be construed as limiting the scope of the invention in any way.

### EXAMPLES

The following Table 2 is referred to throughout the following examples:

TABLE 2
I. LIGATION
Reaction conditions:
95° C. for 2 min
80° C. for 1 min during which Taq ligase is added
25 cycles at 90° C. for 10 sec and 60° C. for 4 min
After cycles, 95° C. for 10 min
Ligation targets

| | |
|---|---|
| 1. COX6b | |
| Ligation probes: | |
| First: | 5′-cctagcgtagtgagcatccgTTGTAGTTCTTGATTTTGG-3′ |
| Second: | 5′-pTCTCCATGTCTTCCGCC [symbol] ATGGCAAGCAGACGTGCGATCTAAtggtagcagtcacgaggcat-Biotin 3′ |
| Template: | 3′-CAGAACATCAAGAACTAAAACCAGAGGTACAGAAGGCGG-5′ |
| Ligation product: | |

5′-cctagcgtagtgagcatccgTTGTAGTTCTTGATTTTGGTCTCCATGTCTTCCGCC [symbol] ATGGCAAGCAGACGTGCGATCTAAtggtagcagtcacgagg

cat-biotin 3′

| | |
|---|---|
| 2. RPS4x | |
| Ligation probes: | |
| First: | 5′-cctagcgtagtgagcatccgtACCCATTTCACCCAC-3′ |
| Second: | 5′-pTGCTCTGTTTGGCCG [symbol] TTCGCGCATAGGAACGCAGAGATtggtagcagtcacgaggcat-biotin 3′ |
| Template: | 3′-TCCCTGGGTAAAGTGGGTGACGAGACAAACCGGCG-5′ |
| Ligation product: | |

5′-cctagcgtagtgagcatccgtACCCATTTCACCACTGCTCTGTTTGGCCG [symbol] TTCGCGCATAGGAACGCAGAGATtggtagcagtcacgaggcat-

biotin 3′

| | |
|---|---|
| 3. GAPDH | |
| Ligation probes: | |
| First: | 5′-cctagcgtagtgagcatccgtAGGGTCTCTCTCTTCC-3′ |
| Second: | 5′-pTCTTGTGCTCTTGCTGG [symbol] CATCGGTCAAGCGGCTGTCCATCAtggtagcagtcacgaggcat-biotin 3′ |
| Template: | 3′-TCACTCCCAGAGAGAGAAGGAGAACACGAGAACGACC-5′ |
| Ligation product: | |

5′-cctagcgtagtgagcatccgtAGGGTCTCTCTCTTCCTCTTGTGCTCTTGCTGG [symbol] CATCGGTCAAGCGGCTGTCCATCAtggtagcagtcacgaggcat

-biotin 3′

| | |
|---|---|
| 4. beta-Actin | |
| Ligation probes: | |
| First: | 5′-cctagcgtagtgagcatccgtATCATCTGGGTCATCTT-3′ |
| Second: | 5′-pCTCGCGGTTGGCCT [symbol] CGCAGGCTCAAGCGATCACTGCtggtagcagtcacgaggcat-biotin 3′ |
| Template | 3′-TTGTACTAGACCCAGTAGAAGAGCGCCAACCGGA-5′ |
| Ligation product: | |

5′-cctagcgtagtgagcatccgtATGATCTGGGTCATCTTCTCGCGGTTGGCCT [symbol] CGCAGGCTCAAGCGATCACTGCtggtagcagtcacgaggcat-

biotin

| | | |
|---|---|---|
| II. A-PCR | | |
| Primers: | Forward primer: | 5′ Cy3-CGGCCCTAGCGTAGTGAGCATCCGT-3′ |
| | Reverse primer: | 5′ ATGCCTCGTGACTGCTAC-3′ |

Reaction Conditions:
95° C. for 10 min
Then, 22 cycles at: 95° C. for 15 sec, 70° C. for 60 sec, 52° C. for 60 sec, and 72° C. for 60 sec;
Then, 72° C. for 7 min;
Then, 25 cycles at: 95° C. for 15 sec, 70° C. for 2 min, and 72° C. for 30 sec;
Then, 95° C. for 10 min and then maintain temperature at 4° C.
III. Quantitation of Ligation or Coupled Ligation-PCR products by TaqMan ™
1. PCR reaction condition:
95° C. for 10 min
40 cycles at 95° C. for 15 sec and 60° C. for 60 sec
2. PCR mixture
Product (1:5 dilution for Ligation;

| | | |
|---|---|---|
| 1:10,000 dilution for Ligation-PCR) | 10 ul | |
| 2X TaqMan Master Mix | 25 ul | |
| Primers: Forward (10 uM) | 4 ul | (Final concentration: 800 nM) |
| Reverse (10 uM) | 4 ul | (Final concentration: 800 nM) |
| TaqMan probe (5 uM) | 2.5 ul | (Final concentration: 250 nM) |
| Distilled water | 4.5 ul | |
| Total | 50 ul | |

| | |
|---|---|
| 3. Target | |
| 3.1. COX6b | |
| TaqMan PCR primers: | |
| Forward: | 5′-CCTAGCGTAGTGAGCATCCGT-3′ |
| Reverse: | 5′-CACGTCTGCTTGCCATGG-3′ |
| TaqMan probe: | 5′ FAM-TGATTTTGGTCTCCATGTCTTCCGCC-TAMRA 3′ |
| 3.2. RPS4x | |
| TaqMan PCR primers: | |
| Forward: | 5′-CCTAGCGTAGTGAGCATCCGT-3′ |
| Reverse: | 5′-GCGTTCCTATGCGCGAA-3′ |
| TaqMan probe: | 5′ FAM-CCATTTCACCCACTGCTCTGTTTGG-TAMRA 3′ |
| 3.3. GAPDR | |
| PCR primers: | |
| Forward: | 5′-CCTAGCGTAGTGAGCATCCGT-3′ |
| Reverse: | 5′-CGCTTGACCGATGCCAG-3′ |
| TaqMan probe: | 5′ FAM-AGGGTCTCTCTCTTCCTCTTGTGCTCTTGC-TAMRA 3′ |
| 3.4. beta-Actin | |
| PCR primers: | |
| Forward: | 5′-CCTAGCGTAGTGAGCATCCGT-3′ |
| Reverse: | 5′-CGCTTGAGCCTGCGAG-3′ |
| TaqMan probe: | 5′ FAM-TGATCTGGGTCATCTTCTCGCGGTTG-TAMRA 3′ |
| IV. Probes deposited on glass slide array | |
| 1. COXEb: | TTAGATCGCACGTCTGCTTGCCAT-Linker-NH2- |
| 2. RPS4X: | AATCTCTGCGTTCCTATGCGCGAA-Linker-NH2- |
| 3. GAPDH: | TGATGGACAGCCGCTTGACCGATG-Linker-NH2- |
| 4. beta-Actin: | CAGCAGTGATCGCTTGAGCCTGCG-Linker-NH2- |

1. Ligation Probe Design

In these examples, a probe set for each target nucleic acid sequence comprised first and second ligation probes designed to adjacently hybridize to the appropriate target nucleic acid sequence. These adjacently hybridized probes were, under appropriate conditions, ligated to form a ligation product. The Tm₅₀ of the first and second probes typically ranged from 42-44° C. at 10 nM.

Multiple potential probe targeting regions were identified for each target nucleic acid sequence, using molding analysis similar to that disclosed in Zuker et al, Algorithms and Thermodynamics for RNA Secondary Structure Prediction: A Practical Guide, in RNA Biochemistry and Biotechnology, pages 11-43, J. Barciszewski & B. F. C. Clark, eds., NATO ASI Series, Kluwer Academic Publishers (1999). Also, Version 3.0 of mfold for Unix operating systems is available via a free license for academic and nonprofit use only; commercial use is available for a fee. Copyright© is held by Washington University.

This illustrative embodiment used four target nucleic acid sequences COX6b, RPS4x, GAPDH (glyceraldehyde-3-phosphate-dehydrogenase), and Beta-actin. Single-stranded cDNA target sequences were used. To assist in selecting appropriate TaqMan™ probes, the potential targeting regions of each target nucleic acid sequence was analyzed using a Primer Express (Primer Express software is available from Applied Biosystems, Foster City, Calif.). Table 2 (I) shows the four nucleic acid sequence templates that were used for the ligation in these examples. Table 2 (III) shows the four Taqman™ probes that were used in these examples. Table 2 (I) shows the four sets of ligation probes that were used in these examples. The ligation probes included a target-specific portion, shown in capital letters in Table 2 (I). As shown in Table 2(I), the ligation probes also included universal priming sequences (21 bases at the 5′ end of the first listed probe in each probe set and 20 bases at the 3′ end of the second listed probe in each probe set). As shown in the boxes in the second listed ligation probe in each probe set in Table 2(I), the ligation probes also included an addressable support-specific portion comprising 24 nucleotides between the target-specific portion of each of the second probes and the primer-specific portion of each of the second probes. In designing the probes, pair-wise comparison was performed to exclude those probes with significant overlapping sequences (4-base perfect matches).

The ligation probes were synthesized using conventional automated DNA synthesis chemistry. Probes were gel-purified prior to use. Typically, 4-20% of polyacrylamide gel is used during purification.

2. Exemplary Ligation Reactions (Oligonucleotide Ligation Assay “OLA”)

In certain embodiments, multiplex ligation reactions may be performed in a 25 μl ligation reaction mixture comprising 20 mM Tris-HCl, pH 7.6, 25 mM potassium acetate, 10 mM magnesium acetate, 10 mM DTT, 1 mM NAD, 0.1% Triton X-100, 2.5-10 nM of each oligonucleotide ligation probe, 0.1 to 1,000 fM of pooled synthetic exemplary target nucleic acid sequences COX6b, RPS4x, GAPDH, and Beta-actin, as shown by the templates in Table 2(I), and 4 to 80 U of thermostable Thermus aquaticus ligase (New England BioLabs, Beverly, Mass.).

In certain embodiments, the ligation reaction mixture is pre-heated at 95° C. for 2 minutes, followed by 80° C. for 1 minute during which Taq ligase is added. Ligation products may then be generated using thermocycling conditions of: 10 to 40 cycles at 90° C. for 10 seconds and 55-60° C. for 4 minutes. After the cycling, in certain embodiments, the mixture is heated at 95° C. for 10-20 minutes. In certain embodiments, the ligation is performed in an ABI 9700 Thermocycler (Applied Biosystems)

3. Exemplary Amplification Reactions

In certain embodiments, ligation products may be diluted (e.g., one to five) and amplified by universal PCR in an ABI 9700 Thermocycler. The Tm₅₀ of the two universal PCR primers used in these examples was designed to differ sufficiently to allow temperature-driven asynchronous PCR (A-PCR), generating an excess of one of the amplification products. The forward primer, which is shown in Table 2(II), had a Tm₅₀ of about 70° C. and was dye-labeled with Cy3 attached to its 5′ end as follows: 5′ Dye-CGGCCCTAGCGTAGTGAGCATCCGT-3′. The reverse primer, which is shown in Table 2(II), had a Tm₅₀ of about 50° C. and had the following sequence: 5′-ATGCCTCGTGACTGCTAC-3′. Thus, at amplification reaction temperatures of approximately 65-70° C., typically no reverse primer was hybridized to the template, while a substantial amount of the forward primer remained hybridized.

In certain embodiments, A-PCR amplification reactions may be performed in 50 μl amplification reaction mixture comprising 10 mM Tris-HCl, pH 8.3, 50 mM KCl, 2-5 mM MgCl₂, 0.01% gelatin, 250 μM of each dNTP, 0.5 to 1 μM forward primer, 0.05 to 0.1 μM reverse primer, 10 μl ligation products (1-1,000 dilution) from Example 2 above, 1-5 U of AmpliTaq Gold DNA polymerase (Applied Biosystems, Foster City, Calif.).

The A-PCR amplification reaction comprises two cycling stages. In certain embodiments, the first cycling stage has an initial denaturation period of 10 minutes at 95° C., followed by 15 to 25 cycles of 95° C. for 15 seconds, 65-70° C. for 60 seconds, 50-55° C. for 60 seconds, and 72° C. for 60 seconds, and an extra extension at 72° C. for 7 minutes. In certain embodiments, the second cycling stage follows immediately and is designed to produce dye-labeled single stranded DNA sequences. In certain embodiments, second stage amplification conditions are 10 to 80 cycles of 95° C. for 15-30 seconds, 66 to 70)° C. for 90 seconds to two minutes, and 70-72° C. for 30-60 seconds. In certain embodiments, those cycles are followed by 95° C. for 10 minutes, followed by maintaining the temperature at 4° C. PCR amplification products may be purified in three washes with distilled water on a Microcon-100 (Millipore, Medford, Mass.).

4. Two Exemplary Coupled Ligation and Amplification Reactions

A first exemplary coupled ligation and amplification reaction (“first exemplary coupled reaction”) was performed. The ligation reaction mixture described in Example 2 was used, and the following components that are described in Example 2 as being included in a range were included in the ligase reaction mixture in the following amounts: 10 nM of each of the 8 oligonucleotide ligation probes as shown in Table 2(I); 100 U of ligase; 100 femtomoles (fM) of each of the four exemplary target nucleic acid sequences, COX6b, RPS4x, GAPDH, and Beta-actin as shown by the templates in Table 2(I).

The ligation reaction mixture was pre-heated at 95° C. for 2 minutes, followed by 80° C. for 1 minute, during which Taq ligase was added. The following conditions were then used: (1) thermocycling using 25 cycles at 90° C. for 10 seconds and 60° C. for 4 minutes, followed by (2) heating at 95° C. for 10 minutes. An ABI 9700 Thermocycler (Applied Biosystems, Foster City, Calif.) was used for the ligation.

The ligation products were then diluted one to five and then amplified in an ABI 9700 Thermocycler (Applied Biosystems, Foster City, Calif.) as follows. The amplification reaction mixture described in Example 3 was used, and the following components that are described in Example 3 as being included in a range were included in the amplification reaction mixture in the following amounts: 2.3 mM MgCl₂; 0.8 μM forward primer as shown in Table 2(II); 0.1 μM reverse primer as shown in Table 2(II); 10 μl ligation products; and 5 U of AmpliTaq Gold DNA polymerase (Applied Biosystems, Foster City, Calif.).

The amplification reaction comprised two cycling stages. The first cycling stage had an initial denaturation period of 10 minutes at 95° C., followed by 22 cycles of 95° C. for 15 seconds, 70° C. for 60 seconds, 52° C. for 60 seconds, and 72° C. for 60 seconds. There was an extra extension at 72° C. for 7 minutes. The second cycling stage followed immediately and was designed to produce dye-labeled single stranded DNA sequences. Amplification conditions for the second stage were 25 cycles of 95° C. for 15 seconds, 70° C. for two minutes, and 72° C. for 30 seconds. Those cycles were followed by 95° C. for 10 minutes, followed by maintaining the temperature at 4° C. PCR amplification products were purified in three washes with distilled water on a Microcon-100 (Millipore, Medford, Mass.).

A second exemplary coupled ligation and amplification reaction (“second exemplary coupled reaction”) was performed. The ligation reaction mixture described in Example 2 was used, and the following components that are described in Example 2 as being included in a range were included in the ligase reaction mixture in the following amounts: 10 nM of each of the 8 oligonucleotide ligation probes shown in Table 2(I); 100 U of ligase; target nucleic acid sequence COX6b (1,000 fM), target nucleic acid sequence RPS4x (100 fM), target nucleic acid sequence GAPDH (10 fM), and target nucleic acid sequence Beta-actin (0.1 fM) (the target nucleic acids were the templates shown in Table 2(I)).

The ligation reaction mixture was pre-heated at 95° C. for 2 minutes, followed by 80° C. for 1 minute, during which Taq ligase was added. The following conditions were then used: (1) thermocycling using 25 cycles at 90° C. for 10 seconds and 60° C. for 4 minutes, followed by (2) heating at 95° C. for 10 minutes. An ABI 9700 Thermocycler (Applied Biosystems, Foster City, Calif.) was used for the ligation.

The ligation products were then diluted one to five and then amplified in an ABI 9700 Thermocycler (Applied Biosystems, Foster City, Calif.) as follows. The amplification reaction mixture described in Example 3 was used, and the following components that are described in Example 3 as being included in a range were included in the amplification reaction mixture in the following amounts: 2.3 mM MgCl₂; 0.8 μM forward primer as shown in Table 2(II); 0.1 μM reverse primer as shown in Table 2(II); 10 μl ligation products; and 5 U of AmpliTaq Gold DNA polymerase (Applied Biosystems, Foster City, Calif.).

The amplification reaction comprised two cycling stages. The first cycling stage had an initial denaturation period of 10 minutes at 95° C., followed by 22 cycles of 95° C. for 15 seconds, 70° C. for 60 seconds, 52° C. for 60 seconds, and 72° C. for 60 seconds. There was an extra extension at 72° C. for 7 minutes. The second cycling stage followed immediately and was designed to produce dye-labeled single stranded DNA sequences. Amplification conditions for the second stage were 25 cycles of 95° C. for 15 seconds, 70° C. for two minutes, and 72° C. for 30 seconds. Those cycles were followed by 95° C. for 10 minutes, followed by maintaining the temperature at 4° C. PCR amplification products were purified in three washes with distilled water on a Microcon-100 (Millipore, Medford, Mass.).

5. Taqman Quantitation

A microarray was used below in Example 6 to quantitate the amplification products produced by the two exemplary coupled ligation and amplification reactions of Example 4. A Taqman™ assay was also used to quantify the ligation products produced in Example 4 and to quantify the amplification products produced by the coupled ligation and amplification reactions of Example 4. The Taqman™ assay is known to those skilled in the art and an exemplary discussion of Taqman™ assay is provided, e.g., in Livak et al., Towards fully automated genome-wide polymorphism screening [letter], Nat Genet, 9(4): p. 341-342 (1995). Amplification primers and double dye-labeled TaqMan™ probes were designed using Primer Express™ (Version 1.0, Applied Biosystems). The Tm₅₀ (the temperature at which only 50% of a nucleic acid species is hybridized to its complement) ranged from 58 to 60° C. for primers and 68 to 70° C. for the TaqMan™ probes, respectively.

In this embodiment, the TaqMan™ probes were designed to be identical to a portion of the ligation product spanning the ligation site. The Taqman™ probes that were used are shown in Table 2(III).

The following Taqman™ amplification reaction mixtures for the Taqman™ assay were used. There was a first set of four different Taqman™ amplification reaction mixtures, and each of the four amplification mixtures comprised 10 microliters of a 1:5 dilution of products from the first ligation reaction of Example 4 in which 100 fM of each of the four target nucleic acid sequences were used. Each of the four different amplification reaction mixtures further comprised one of the four different Taqman™ primer sets and Taqman™ probes for each of the four different target nucleic sequences as shown in Table 2(III).

There was also a second set of four different Taqman™ amplification reaction mixtures, and each of the four amplification mixtures comprised 10 microliters of a 1:5 dilution of products from the second ligation reaction of Example 4 in which varying amounts of the four target nucleic acid sequences were used. Each of the four different amplification reaction mixtures further comprised one of the four different Taqman™ primer sets and Taqman™ probes for each of the four different target nucleic sequences as shown in Table 2(III).

There was also a third set of four different Taqman™ amplification reaction mixtures, and each of the four amplification mixtures comprised 10 microliters of a 1 :10,000 dilution of products from the first exemplary coupled ligation and amplification reaction of Example 4 in which 100 fM of each of the four target nucleic acid sequences were used. Each of the four different amplification reaction mixtures further comprised one of the four different Taqman™ primer sets and Taqman™ probes for each of the four different target nucleic sequences as shown in Table 2(III).

There was also a fourth set of four different Taqman™ amplification reaction mixtures, and each of the four amplification mixtures comprised 10 microliters of a 1:10,000 dilution of products from the second exemplary coupled ligation and amplification reaction of Example 4 in which varying amounts of the four target nucleic acid sequences were used. Each of the four different amplification reaction mixtures further comprised one of the four different Taqman™ primer sets and Taqman™ probes for each of the four different target nucleic sequences as shown in Table 2(III).

Thus, there were 16 different Taqman™ amplification reaction mixtures. Each of the sixteen different Taqman™ amplification reaction mixtures comprised 4 μl (final concentration 800 nM) of the forward primer (10 μM), 4 μl (final concentration 800 nM) of the reverse primers (10 μM), and 2.5 μl (final concentration 250 nM) of each of the Taqman™ probe (5 μM) for the given target to be detected in each reaction mixture.

Each of the sixteen different Taqman™ amplification reaction mixtures further comprised 2×Taqman™ Master mix (Applied Biosystems, Foster City, Calif.) (25 μl). The Taqman™ Master mix includes PCR buffer, dNTPs, MgCl₂, and AmpliTaq Gold DNA polymerase (Applied Biosystems, Foster City, Calif.). Each of the sixteen Taqman™ amplification reaction mixtures further comprised 4.5 μl of distilled water. Each of the sixteen different Taqman™ reaction mixtures was run in triplicate so there were 48 reaction containers that each contained 50 μl of amplification reaction mixture.

The Taqman™ amplification reaction was performed as follows. The amplification reaction mixtures were heated at 95° C. for 10 minutes. The thermal cycling was performed with 40 cycles of 95° C. for 15 seconds and 60° C. for 1 minute. All reactions were performed in an ABI 7700 Sequence Detector (Applied Biosystems, Foster City, Calif.). Reaction conditions were programmed on a Power Macintosh G3 computer (Apple Computer, Cupertino, Calif.) linked directly to an ABI 7700 Sequence Detector (Applied Biosystems). Analysis of data was also performed on the Power Macintosh G3 computer, using data collection and analysis software developed by Applied Biosystems (SDS Analysis V3.7).

FIG. 12 shows the results of the Taqman™ assay of the ligation products and the amplification products of the first exemplary coupled reaction described in Example 4 above. In FIG. 12, the X axis shows the number of cycles and the Y axis shows the change in fluorescence signal (delta Rn). As shown in FIG. 12, when the ligation products (FIG. 12A) and the amplification products (FIG. 12B) for the four target nucleic acid sequences from the first exemplary coupled reaction were quantitated by the TaqMan™ assay, performed as described above, each of the four ligation products appeared at substantially the same rate, and each of the four amplification products appeared at substantially the same rate, as seen by the parallel, substantially superimposed, ligation product and amplification product curves. Thus, quantitative results were observed when equimolar concentrations of four individual target nucleic acid sequences were used.

FIG. 13 shows the results of the Taqman™ assay of the ligation products and the amplification products of the second exemplary coupled reaction described in Example 4 above. In FIG. 13, the X axis shows the number of cycles and the Y axis shows the change in fluorescence signal (delta Rn). As shown in FIG. 13, when the ligation products (FIG. 13A) and amplification products (FIG. 13B) for the four target templates from the second exemplary coupled reaction were quantitated by TaqMan™ assay, performed as described above, the four ligation products appeared at the different rates, and the four amplification products appeared at the different rates, dependent on the initial target nucleic acid sequence concentration.

6. Exemplary Microarray Generation, Hybridization, Detection and Analysis

Another portion of the amplification products from the two exemplary coupled reactions in Example 4 were exposed to microarrays rather than the TaqMan™ assay described in Example 5 above.

Microarrays were generated on one inch by 3 inch glass slides using capture oligonucleotides that were attached using a 5′-amino linker. A total of 64 different 24-mer oligonucleotides array probes with eight replicates of each of the 64 different 24-mer probes were spotted on glass slides (64×8=512 locations on the slide). Table 2(IV) shows four of the 64 different probes deposited on the glass slide array, which had sequences for hybridizing to the addressable support-specific portions of the amplified ligation products of the two exemplary coupled ligation and amplification reactions of Example 4.

Two separate hybridization reaction mixtures were prepared. The first hybridization reaction mixture comprised 2 μl of the PCR amplification product from the first exemplary coupled reaction in Example 4 above, 25 μl 4×SSC, 0.3% SDS, 1 μg/μl yeast tRNA, 1 μg/μl poly(A). The second hybridization reaction mixture comprised 2 μl of the PCR amplification product from the second exemplary coupled reaction in Example 4 above, 25 μl 4×SSC, 0.3% SDS, 1 μg/μl yeast tRNA, 1 μg/μl poly(A).

Each to the two hybridization mixtures was separately denatured at 95° C. for 2 to 4 minutes, then the separate denatured mixtures were separately applied to two microarray slides for each of the two hybridization mixtures (four slides). The slides were placed inside a sealed array chamber with a drop of buffer to reduce or prevent evaporation. Following hybridization at 50-55° C. in a waterbath for 16-20 hours, the microarray slides were washed briefly in 500 ml of 4×SSC containing 0.3% SDS at 50-55° C., washed once for 2 minutes in 500 ml of 1×SSC containing 0.3% SDS at room temperature, followed by two washes in 500 ml of 0.06×SSC at room temperature for 2 minutes each. Microarrays were imaged using an Axon scanner, and images were analyzed in GenePix Pro 3.0 software (Axon Instruments, Foster City, Calif.).

Using this procedure, the amplification products of the first and second exemplary coupled reactions were detected and analyzed. As shown in FIG. 14, the amplification products of the first exemplary coupled reaction, wherein the initial concentration of the four target nucleic acid sequences was equimolar, provided similar signal intensities. The four quantitated signals ranged from 10,536 (±8,080) for COX6b to 13,153 (±6,922) for GAPDH. Thus, the coupled ligation and amplification reaction comprising a universal primer set provided quantitatively similar results under these conditions.

The amplification products of the second exemplary coupled reaction, wherein the initial concentration for the four templates varied, provided relatively quantitative results under these conditions. As shown in FIG. 15, the signal intensity for the four targets ranged from 28,159 (±16,584) for COX6b (initial template concentration of 1000 fM) to 1,969 (±714) for Beta-actin (initial template concentration of 0.1 fM). Thus, under these conditions, the signal intensity that was detected and quantitated varied with the initial template concentration.

Although the invention has been described with reference to certain applications, methods, and compositions, it will be appreciated that various changes and modifications may be made without departing from the invention.

What is claimed is:

1. A method for quantitating at least one target nucleic acid sequence in a sample comprising:
combining at least one target nucleic acid sequence with a probe set for each target nucleic acid sequence, the probe set comprising (a) at least one first probe, comprising a first target-specific portion, and (b) at least one second probe, comprising a second target-specific portion and a 3′ primer-specific portion, wherein the probes in each set are suitable for ligation together when hybridized adjacent to one another on the at least one target nucleic acid sequence, and wherein at least one probe in each probe set further comprises at least one addressable support-specific portion, and when the at least one first probe comprises the at least one addressable support-specific portion, the at least one first probe further comprises a 5′ primer-specific portion, and wherein the at least one addressable support-specific portion is located between the primer-specific portion and the target-specific portion of the at least one probe in each probe set; to form a ligation reaction mixture;
subjecting the ligation reaction mixture to at least one cycle of ligation, wherein adjacently hybridized probes are ligated to form a ligation product comprising the first and second target specific portions, the at least one addressable support-specific portion, and the 3′ primer-specific portion;
combining the ligation product with at least one primer set comprising at least one second primer comprising a sequence complementary to the 3′ primer-specific portion of the ligation product and a DNA polymerase; to form a first amplification reaction mixture;
subjecting the first amplification reaction mixture to at least one cycle of amplification to generate a first amplification product;
detecting the first amplification product or a portion of the first amplification product using the at least one addressable support-specific portion; and
quantitating the at least one target nucleic acid sequence.

2. The method of claim 1, wherein the at least one first probe further comprises a 5′ primer-specific portion, wherein the ligation product further comprises the 5′ primer-specific portion, and wherein the at least one primer set further comprises at least one first primer comprising the sequence of the 5′ primer-specific portion.

3. The method of claim 2, wherein the first amplification product further comprises a reporter group, and wherein the quantitating further comprises determining the amount of the at least one reporter group.

4. The method of claim 3 wherein the at least one target nucleic acid sequence comprises at least one complementary DNA (cDNA) generated from an RNA.

5. The method of claim 4, wherein the at least one cDNA is generated from a messenger RNA (mRNA).

6. The method of claim 3, wherein the at least one target nucleic acid sequence comprises at least one RNA.

7. The method of claim 6, wherein the ligation reaction mixture further comprises at least one of a T4 DNA ligase, a T7 DNA ligase, or an enzymatically active mutant or variant thereof.

8. The method of claim 3, wherein the detecting comprises hybridizing the addressable support-specific portion of the first amplification product or a portion of the first amplification product comprising at least one reporter group directly or indirectly to a support.

9. The method of claim 8, further comprising denaturing the first amplification product to generate single-stranded portions of the amplification product.

10. The method of claim 9, wherein the denaturing comprises heating the amplification product to a temperature above the melting temperature of the amplification product to generate single-stranded portions.

11. The method of claim 9, wherein the denaturing comprises chemically denaturing the amplification product to generate single-stranded portions.

12. The method of claim 8, wherein the first probe further comprises the addressable support-specific portion.

13. The method of claim 8, wherein the second probe further comprises the addressable support-specific portion.

14. The method of claim 1, wherein the addressable support-specific portion comprises a mobility modifier sequence.

15. The method of claim 14, wherein the mobility modifier sequence is less than 101 nucleotides in length.

16. The method of claim 15, wherein the mobility modifier sequence is less than 41 nucleotides in length.

17. The method of claim 15, wherein the mobility modifier sequence is 2-36 nucleotides in length.

18. The method of claim 14, wherein the first probe further comprises the mobility modifier sequence.

19. The method of claim 14, wherein the second probe further comprises the mobility modifier sequence.

20. The method of claim 14, wherein the detecting comprises subjecting the first amplification product or a portion of the first amplification product comprising at least one reporter group to a procedure for separating nucleic acid sequences based on molecular weight or length.

21. The method of claim 20, wherein the separating comprises at least one mobility-dependent analysis technique (MDAT).

22. The method of claim 21, wherein the MDAT comprises at least one of electrophoresis, chromatography, HPLC, mass spectroscopy, sedimentation, field-flow fractionation, or multi-stage fractionation.

23. The method of claim 22, wherein the MDAT comprises electrophoresis.

24. The method of claim 20, wherein the separating comprises dialyzing the first amplification product or a portion of the first amplification product comprising at least one reporter group.

25. The method of claim 1, wherein the ligation reaction mixture further comprises a ligation agent.

26. The method of claim 25, wherein the ligation agent is a ligase.

27. The method of claim 26, wherein the ligase is a thermostable ligase.

28. The method of claim 27, wherein the thermostable ligase is at least one of Tth ligase, Taq ligase, Tsc ligase, Pfu ligase, and an enzymatically active mutant or variant thereof.

29. The method of claim 1, wherein the DNA polymerase is a thermostable polymerase.

30. The method of claim 29, wherein the thermostable DNA polymerase is at least one of Taq polymerase, Pfx polymerase, Pfu polymerase, Vent® polymerase, Deep Vent™ polymerase, Pwo polymerase, Tth polymerase, and an enzymatically active mutant or variant thereof.

31. The method of claim 1, wherein the reporter group comprises a fluorescent moiety.

32. The method of claim 2, wherein the melting temperature of the at least one first primer differs from the melting temperature of the at least one second primer by at least about 8° C. in at least one primer set.

33. The method of claim 2, wherein the first amplification product comprises at least one 5′ terminal phosphate; and further comprising:
combining the first amplification product with an exonuclease to form a digestion reaction mixture; and
incubating the digestion reaction mixture under conditions that allow the exonuclease to digest the amplification product to generate a portion of the first amplification product comprising at least one reporter group.

34. A method for quantitating at least one target nucleic acid sequence in a sample comprising:
combining at least one target nucleic acid sequence with a probe set for each target nucleic acid sequence, the probe set comprising (a) at least one first probe, comprising a first target-specific portion, and (b) at least one second probe, comprising a second target-specific portion and a 3′ primer-specific portion, wherein the probes in each set are suitable for ligation together when hybridized adjacent to one another on the at least one target nucleic acid sequence, and wherein at least one probe in each probe set further comprises a promoter or its complement, and wherein at least one probe in each probe set further comprises at least one addressable support-specific portion, and when the at least one first probe comprises the at least one addressable support-specific portion, the at least one first probe further comprises a 5′ primer-specific portion, and wherein the at least one addressable support-specific portion is located between the primer-specific portion and the target-specific portion of the at least one probe in each probe set; to form a ligation reaction mixture;
subjecting the ligation reaction mixture to at least one cycle of ligation, wherein adjacently hybridized probes are ligated to form a ligation product comprising the first and second target specific portions, the at least one addressable support-specific portion, the 3′ primer-specific portion, and the promoter sequence or its complement;
combining the ligation product with at least one primer set comprising at least one second primer comprising a sequence complementary to the 3′ primer-specific portion of the ligation product and a DNA polymerase, to form a first amplification reaction mixture;
subjecting the first amplification reaction mixture to at least one cycle of amplification to generate a first amplification product comprising the promoter sequence;
combining the first amplification product with an RNA polymerase and a ribonucleoside triphosphate solution comprising at least one of rATP, rCTP, rGTP, or rUTP, to form a transcription reaction mixture;
incubating the transcription reaction mixture under appropriate conditions to generate an RNA transcription product;
detecting the RNA transcription product or a portion of the RNA transcription product using the at least one addressable support-specific portion; and
quantitating the at least one target nucleic acid sequence.

35. The method of claim 34, wherein the at least one first probe further comprises a 5′ primer-specific portion, wherein the ligation product further comprises the 5′ primer-specific portion, and wherein the at least one primer set further comprises at least one first primer comprising the sequence of the 5′ primer-specific portion.

36. The method of claim 35, wherein the at least one ribonucleoside triphosphate further comprises a reporter group, and wherein the quantitating further comprises determining the amount of the at least one reporter group.

37. The method of claim 36, wherein the at least one target nucleic acid sequence comprises at least one complementary DNA (cDNA) generated from an RNA.

38. The method of claim 37, wherein the at least one cDNA is generated from a messenger RNA (mRNA).

39. The method of claim 36, wherein the at least one target nucleic acid sequence comprises at least one RNA.

40. The method of claim 39, wherein the ligation reaction mixture further comprises at least one of a T4 DNA ligase and an enzymatically active mutant or variant thereof.

41. The method of claim 36, wherein the detecting comprises hybridizing the addressable support-specific portion of the RNA transcription product or a portion of the RNA transcription product directly or indirectly to a support.

42. The method of claim 41, wherein the first probe further comprises the addressable support-specific portion.

43. The method of claim 41, wherein the second probe further comprises the addressable support-specific portion.

44. The method of claim 36, wherein the addressable support-specific portion comprises a mobility modifier sequence.

45. The method of claim 44, wherein the mobility modifier sequence is less than 101 nucleotides in length.

46. The method of claim 45, wherein the mobility modifier sequence is less than 41 nucleotides in length.

47. The method of claim 45, wherein the mobility modifier sequence is 2-36 nucleotides in length.

48. The method of claim 44, wherein the first probe further comprises the mobility modifier sequence.

49. The method of claim 44, wherein the second probe further comprises the mobility modifier sequence.

50. The method of claim 44, wherein the detecting comprises subjecting the RNA transcription product to a procedure for separating nucleic acid sequences based on molecular weight or length.

51. The method of claim 50, wherein the separating comprises at least one MDAT.

52. The method of claim 51, wherein the MDAT comprises at least one of electrophoresis, chromatography, HPLC, mass spectroscopy, sedimentation, field-flow fractionation, and multi-stage fractionation.

53. The method of claim 52, wherein the MDAT comprises electrophoresis.

54. The method of claim 50, wherein the separating comprises dialyzing the RNA transcription products.

55. The method of claim 36, wherein the ligation reaction mixture further comprises a ligation agent.

56. The method of claim 55, wherein the ligation agent is a ligase.

57. The method of claim 56, wherein the ligase is a thermostable ligase.

58. The method of claim 57, wherein the thermostable ligase is at least one of Tth ligase, Taq ligase, Tsc ligase, Pfu ligase, and an enzymatically active mutant or variant thereof.

59. The method of claim 36, wherein the thermostable DNA polymerase is a thermostable polymerase.

60. The method of claim 59, wherein the DNA polymerase is at least one of Taq polymerase, Pfx polymerase, Pfu polymerase, Vent® polymerase, Deep Vent™ polymerase, Pwo polymerase, Tth polymerase, and an enzymatically active mutant or variant thereof.

61. The method of claim 36, wherein the reporter group comprises a fluorescent moiety.

62. The method of claim 36, wherein the RNA polymerase is at least one of pho RNA polymerase, bacteriophage T3 RNA polymerase, T7 RNA polymerase, SP6 RNA polymerase, and an enzymatically active mutant or variant thereof.

63. The method of claim 36, wherein the promoter is upstream of the addressable support-specific portion.

64. A method for quantitating at least one target nucleic acid sequence in a sample comprising:
combining at least one target nucleic acid sequence with a probe set for each target nucleic acid sequence, the probe set comprising (a) a first probe, comprising a first target-specific portion and a 5′ primer-specific portion, and (b) a second probe, comprising a second target-specific portion and a 3′ primer-specific portion, wherein the probes in each set are suitable for ligation together when hybridized adjacent to one another on the at least one target nucleic acid sequence, and wherein at least one probe in each probe set further comprises at least one addressable support-specific portion located between the primer-specific portion and the target-specific portion of the at least one probe in each probe set; to form a ligation reaction mixture;
subjecting the ligation reaction mixture to at least one cycle of ligation, wherein adjacently hybridized probes are ligated to one another to form a ligation product comprising the 5′ primer-specific portion, the first and second target specific portions, the at least one addressable support-specific portion, and the 3′ primer-specific portion;
combining the ligation product with: (a) at least one primer set comprising: (i) at least one first primer comprising the sequence of the 5′ primer-specific portion of the ligation product, and (ii) at least one second primer comprising a sequence complementary to the 3′ primer-specific portion of the ligation product; and (b) a DNA polymerase; to form a first amplification reaction mixture;
subjecting the first amplification reaction mixture to at least one cycle of amplification to generate a first amplification product;
combining the first amplification product with either at least one first primer, or at least one second primer for each primer set, but not both first and second primers, to form a second amplification reaction mixture;
subjecting the second amplification reaction mixture to at least one cycle of amplification to generate a second amplification product;
detecting the second amplification product or a portion of the second amplification product using the at least one addressable support-specific portion; and
quantitating the expression of the at least one target nucleic acid sequence.

65. The method of claim 64, wherein the at least one amplification product further comprises a reporter group, and wherein the quantitating further comprises determining the amount of the at least one reporter group.

66. The method of claim 65, wherein the at least one target nucleic acid sequence comprises at least one complementary DNA (cDNA) generated from an RNA.

67. The method of claim 66, wherein the at least one cDNA is generated from an mRNA.

68. The method of claim 65, wherein the at least one target nucleic acid sequence comprises at least one RNA.

69. The method of claim 68, wherein the ligation reaction mixture further comprises at least one of a T4 DNA ligase and an enzymatically active mutant or variant thereof.

70. The method of claim 65, wherein the detecting comprises hybridizing the addressable support-specific portion of the second amplification product or a portion of the second amplification product directly or indirectly to a support.

71. The method of claim 65, wherein the first probe further comprises the addressable support-specific portion.

72. The method of claim 65, wherein the second probe further comprises the addressable support-specific portion.

73. The method of claim 65, wherein the addressable support-specific portion comprises a mobility modifier sequence.

74. The method of claim 73, wherein the mobility modifier sequence is less than 101 nucleotides in length.

75. The method of claim 74, wherein the mobility modifier sequence is less than 41 nucleotides in length.

76. The method of claim 74, wherein the mobility modifier sequence is 2-36 nucleotides in length.

77. The method of claim 73, wherein the first probe further comprises the mobility modifier sequence.

78. The method of claim 73, wherein the second probe further comprises the mobility modifier sequence.

79. The method of claim 73, wherein the detecting comprises subjecting the second amplification product to a procedure for separating nucleic acid sequences based on molecular weight or length.

80. The method of claim 79, wherein the separating comprises at least one MDAT.

81. The method of claim 80, wherein the MDAT comprises at least one of electrophoresis, chromatography, HPLC, mass spectroscopy, sedimentation, field-flow fractionation, or multi-stage fractionation.

82. The method of claim 80, wherein the MDAT comprises electrophoresis.

83. The method of claim 79, wherein the separating comprises dialyzing the second amplification product.

84. The method of claim 64, wherein the ligation reaction mixture further comprises a ligation agent.

85. The method of claim 84, wherein the ligation agent is a ligase.

86. The method of claim 85, wherein the ligase is a thermostable ligase.

87. The method of claim 86, wherein the thermostable ligase is at least one of Tth ligase, Taq ligase, Tsc ligase, Pfu ligase, and an enzymatically active mutant or variant thereof.

88. The method of claim 65, wherein the DNA polymerase is a thermostable polymerase.

89. The method of claim 88, wherein the thermostable DNA polymerase is at least one of Taq polymerase, Pfx polymerase, Pfu polymerase, Vent® polymerase, Deep Vent™ polymerase, Pwo polymerase, Tth polymerase, and an enzymatically active mutant or variant thereof.

90. The method of claim 65, wherein the reporter group comprises a fluorescent moiety.

91. A kit for quantitating the expression of at least one target nucleic acid sequence comprising:
at least one probe set for each target nucleic acid sequence to be detected, the probe set comprising (a) at least one first probe, comprising a first target-specific portion and a 5′ primer-specific portion, and (b) at least one second probe, comprising a second target-specific portion and a 3′ primer-specific portion, wherein the probes in each probe set are suitable for ligation together when hybridized adjacent to one another on the at least one target nucleic acid sequence, and wherein at least one probe in each probe set further comprises at least one addressable support-specific portion located between the primer-specific portion and the target-specific portion of the at least one probe in each probe set.

92. A kit according to claim 91, further comprising a DNA polymerase.

93. A kit according to claim 92, wherein the DNA polymerase is thermostable.

94. A kit according to claim 93, wherein the thermostable polymerase is at least one of Taq polymerase, Pfx polymerase, Pfu polymerase, Vent® polymerase, Deep Vent™ polymerase, Pwo polymerase, Tth polymerase, and an enzymatically active mutant or variant thereof.

95. A kit according to claim 91, further comprising a set of primers, the primer set comprising (i) at least one primer comprising the sequence of the 5′ primer-specific portion of the first probe, and (ii) at least one primer comprising a sequence complementary to the 3′ primer-specific portion of the second probe, wherein at least one primer of the primer set further comprises a reporter group.

96. A kit according to claim 95, further comprising a DNA polymerase.

97. A kit according to claim 96, wherein the DNA polymerase is thermostable.

98. A kit according to claim 97, wherein the thermostable polymerase is at least one of Taq polymerase, Pfx polymerase, Pfu polymerase, Vent® polymerase, Deep Vent™ polymerase, Pwo polymerase, Tth polymerase, and an enzymatically active mutant or variant thereof.

99. A kit according to claim 91, wherein the addressable support-specific portion of at least one probe comprises a mobility modifier sequence.

100. A kit according to claim 91, further comprising a support, the support comprising capture oligonucleotides capable of hybridizing with addressable support-specific portion of the at least one probe or with a sequence complementary to the addressable support-specific portion of the at least one probe.

101. A kit according to claim 91, further comprising a ligase.

102. A kit according to claim 101, wherein the ligase is T4 DNA ligase.

103. A kit according to claim 101, wherein the ligase is thermostable.

104. A kit according to claim 103, wherein the thermostable ligase is at least one of Tth ligase, Taq ligase, Pfu ligase, and an enzymatically active mutant or variant thereof.

105. A kit according to claim 91, wherein at least one probe in each probe set further comprises a promoter sequence or its complement.

106. A kit according to claim 105, further comprising a RNA polymerase.

107. A kit according to claim 106, wherein the RNA polymerase is at least one of a pho RNA polymerase, bacteriophage T3 RNA polymerase, T7 RNA polymerase, SP6 RNA polymerase, and an enzymatically active mutant or variant thereof.

108. A kit according to claim 106, wherein the RNA polymerase is thermostable.

109. A kit according to claim 91, wherein the first probe of each probe set further comprises a phosphorothioate group at the 3′-end.

110. A kit according to claim 91, wherein the second probe of each probe set further comprises a 5′ thymidine residue with a leaving group suitable for ligation.

111. A kit according to claim 110, wherein the 5′ thymidine leaving group is tosylate or iodide.

112. A kit according to claim 91, wherein the first probe of each probe set further comprises a phosphorothioate group at the 3′-end and wherein the second probe of each probe set further comprises a 5′ thymidine residue with a leaving group suitable for ligation.

113. A kit according to claim 112, wherein the 5′ thymidine leaving group is tosylate or iodide.

114. A kit for quantitating the expression of at least one target nucleic acid sequence comprising:
at least one probe set for each target nucleic acid sequence to be detected, each probe set comprising (a) a first probe, comprising a first target-specific portion and (b) a second probe, comprising a second target-specific portion and a 3′ primer-specific portion, wherein the probes in each set are suitable for ligation together when hybridized adjacent to one another on the at least one target nucleic acid sequence, and wherein at least one second probe in each probe set further comprises at least one addressable support-specific portion located between the primer-specific portion and the target-specific portion of the at least one second probe in each probe set.

115. A kit according to claim 114, wherein the addressable support-specific portion comprises a mobility modifier sequence.

116. A kit according to claim 114, further comprising a support, the support comprising capture oligonucleotides capable of hybridizing with addressable support-specific portion of the at least one probe or with a sequence complementary to the addressable support-specific portion of the at least one probe.

117. A kit according to claim 114, further comprising a primer set comprising at least one primer complementary to the 3′ primer-specific portion of the second probe, wherein at least one primer of the primer set further comprises a reporter group; and a DNA polymerase.

118. A kit according to claim 117, wherein the reporter group comprises a fluorescent moiety.
